(19) United States
(12) Patent Application Publication
(10) Pub. No.: US 2025/0235399 A1
(43) Pub. Date: Jul. 24, 2025

(54) METHODS OF PREPARING CARRIER-BASED DRY POWDERS FOR INHALATION

(71) Applicant: Board of Regents, The University of Texas System, Austin, TX (US)

(72) Inventors: Hugh Smyth, Austin, TX (US); Feng Zhang, Austin, TX (US); Jamie Spahn, Austin, TX (US)

(73) Assignee: Board of Regents, The University of Texas System, Austin, TX (US)

(21) Appl. No.: 18/849,399

(22) PCT Filed: Apr. 7, 2023
(86) PCT No.: PCT/US2023/017982

§ 371 Date: Sep. 20, 2024

Related U.S. Application Data
(60) Provisional application No. 63/328,440, filed on Apr. 7, 2022.

(51) Int. Cl. A61K 9/00; A61K 9/14; A61K 31/496; A61K 31/58

(52) U.S. Cl. CPC A61K 9/0075; A61K 9/145; A61K 31/496; A61K 31/58

(57) ABSTRACT

Described herein are dry powder inhalation mixtures for pulmonary delivery and processes for making the same. A process for making a dry powder inhaler blend as described herein comprises introducing an active pharmaceutical ingredient, a lubricant, and a carrier into a multi-screw extruder; and continuously mixing the active pharmaceutical ingredient, the lubricant, and the carrier in the multi-screw extruder to form a dry powder inhaler blend. In this method, the active pharmaceutical ingredient, the lubricant, and the carrier are in the form of a powder.

### CROSS REFERENCE TO RELATED APPLICATIONS

This application claims the benefit of and priority to U.S. Provisional Application No. 63/328,440, filed on Apr. 7, 2022, which is hereby incorporated by reference in its entirety.

### BACKGROUND

Dry powder inhalation systems have been successfully used for modem respiratory drug delivery for over 50 years since the introduction of the Spinhaler™ by Fisons Pharmaceuticals in 1971. In recent years, they have become a predominant technology platform chosen to deliver new therapies due to their previously described advantages. Most of these marketed products rely on interactive carrier blend formulations. Typically in these mixtures, a relatively low concentration of micronized drug particles are blended with coarser lactose particles that comprise the bulk of the formulation. The advantages of utilizing lactose carrier formulations are limited by the difficulty of achieving stable homogenous mixtures that also confer optimal dispersion and aerosolization performance. Indeed, various processing approaches to achieving dry powder inhalation blends have been described and include low shear blending, high shear blending, and also using ternary blends in which a third component is added to facilitate desired blending uniformity and/or aerosol performance.

Despite the availability of various manufacturing and formulation strategies for dry powder inhalers, even successfully marketed products suffer from batch-to-batch variability. The exact causes of batch-to-batch variability has not been elucidated but are likely multicomponent. Such possible causes include, for example, significant effects of mild compression forces experienced during batch powder manufacturing processes on the flowability and aerosol performance of a lactose-based dry powder inhaler formulation. These observed batch-to-batch variations in dry powder inhaler (DPI) aerosol performance may result in significant pharmacokinetic differences as observed between batches of marketed products. Despite this well-known and critical issue, the industry continues to utilize historical batch manufacturing processes for these products.

### SUMMARY

Described herein are dry powder inhalation mixtures for pulmonary delivery and processes for making the same. An example process for making a dry powder inhaler blend as described herein comprises introducing an active pharmaceutical ingredient, a lubricant, and a carrier into a multi-screw extruder; and continuously mixing the active pharmaceutical ingredient, the lubricant, and the carrier in the multi-screw extruder to form a dry powder inhaler blend. In this method, the active pharmaceutical ingredient, the lubricant, and the carrier are in the form of a powder, for example. The multi-screw extruder may comprise at least two co-rotating screws and an intermeshing region. The intermeshing region may be positioned between the at least two co-rotating screws. Optionally, the multi-screw extruder can be operated at a screw speed of rotation of at least 50 rpm (e.g., from 50 rpm to 1000 rpm). In some cases, a feed rate of the components into the multi-screw extruder is from 1 g/min to 50 g/min.

In some cases, the dry powder inhaler blend has a coefficient of variation percent (% CV) value of less than 5% for any combination of screw speed of rotation of the extruder and feed rate of the components into the extruder. In some cases, the % CV value is less than 5% where the screw speed is 50 rpm and the feed rate is 10 g/min. In some cases, the % CV value is less than 5% where the screw speed is 100 rpm and the feed rate is 20 g/min. In some cases, the % CV value is less than 5% where the screw speed is 200 rpm and the feed rate is 15 g/min. In some cases, the % CV value is less than 5% where the screw speed is 200 rpm and the feed rate is 25 g/min. In some cases, the % CV value is less than 5% where the screw speed is 200 rpm and the feed rate is 40 g/min. In some cases, the % CV value is less than 5% where the screw speed is 300 rpm and the feed rate is 10 g/min. In some cases, the % CV value is less than 5% where the screw speed is 350 rpm and the feed rate is 40 g/min. In some cases, the % CV value is less than 5% where the screw speed is 500 rpm and the feed rate is 10 g/min. In some cases, the % CV value is less than 5% where the screw speed is 500 rpm and the feed rate is 40 g/min.

Also disclosed herein is a capsule for an inhaler, such as comprising a dry powder inhaler blend comprising an active pharmaceutical ingredient, a lubricant, and a carrier. The dry powder inhaler blend can be prepared according to a method as described herein. Optionally, the active pharmaceutical ingredient comprises an agent suitable for treating a pulmonary disease or a pulmonary infection (e.g., an antibacterial agent, a steroid, or any other agent suitable for treating a pulmonary disease or infection). Optionally, the active pharmaceutical ingredient comprises an agent for administration by inhalation.

Further described herein are dry powder inhaler blends. In some examples, a dry powder inhaler blend comprises rifampicin in an amount of 5 wt. % or less, a lubricant, and a carrier, wherein the % CV value is less than 5%. In some examples, a dry powder inhaler blend comprises budesonide in an amount of 5 wt. % or less, a lubricant, and a carrier, wherein the % CV value is less than 5%. Optionally, the lubricant is magnesium stearate and is optionally present in an amount of 1 wt. % or less. Optionally, the carrier is lactose. The dry powder inhaler blend can be prepared by continuous mixing in a multi-screw extruder, for example, according to the methods described herein.

Further described herein are processes for making a low dose dry powder blend, such as comprising introducing an active pharmaceutical ingredient, a lubricant, and a carrier into at least one multi-crew extruder and continuously mixing the active pharmaceutical ingredient, the lubricant, and the carrier in the multi-screw extruder to form a low dose dry powder blend, wherein the active pharmaceutical ingredient, the lubricant, and the carrier are in the form of a powder and, wherein the pharmaceutical ingredient is at most 5% of the low dose dry powder blend by weight.

Also disclosed herein are dosage forms, such as comprising a dry powder blend comprising an active pharmaceutical ingredient, a lubricant, and a carrier. The dry powder blend can be prepared according to a method as described herein. Optionally, the active pharmaceutical ingredient comprises an agent suitable for treating one or more diseases or infections selected from the group consisting of a pulmonary disease or a pulmonary infection, a cardiac disease or cardiac infection, a gastrointestinal disease or gastrointestinal infection, a dermal disease or dermal infection, an epidermal disease or epidermal infection, a muscular disease or muscular infection, a skeletal disease or skeletal infection, a lymphatic disease or lymphatic infection, or a blood disease or blood infection. Optionally, the agent suitable for treating a disease or infection comprises an antibacterial agent or a steroid.

The details of one or more embodiments are set forth in the drawings and the description below. Other features, objects, and advantages will be apparent from the description and drawings, and from the claims.

### BRIEF DESCRIPTION OF THE DRAWINGS

FIG. 1A illustrates a continuous mixing screw profile. FIG. 2B illustrates a longitudinal depiction of the GMF-3-15-30 element. FIG. 1C illustrates a cross-sectional depiction of the GMF-3-15-30 element).

FIG. 2A is a bar graph showing blend uniformity of 1% rifampicin with 0.4% magnesium stearate in lactose prepared by twin-screw extruder. FIG. 2B is a bar graph showing blend uniformity of 10% rifampicin with 0.4% magnesium stearate in lactose prepared by low-shear mixer.

FIG. 3A is a scattered plot of the coefficient of variation (CV) for content uniformity for twin-screw blends. FIG. 3B is a scattered plot of the coefficient of variation (CV) for content uniformity for low-shear blends.

FIG. 4 is a collection of bar graphs depicting aerosol performance as measured using the fine particle fraction (FPF) for rifampicin blends prepared using twin-screw mixing compared to low-shear mixing.

FIG. 5 is a bar graph illustrating rifampicin retention in the blending device and capsule during next generation impactor (NGI) measurements for twin-screw blended powders and a low-shear mixer blended powder.

FIG. 6 is a graph of the geometric particle size of the rifampicin powder blends as measured using laser diffraction (HELOS) using the RODOS powder disperser at a pressure drop of 1 bar and rotor speed set at 50%.

FIG. 7A is a powder x-ray diffraction (PXRD) data plot of starting materials and the final extruder blend showing no detectable changes in rifampicin or lactose crystallinity or physical form. FIG. 7B is a differential scanning calorimetry (DCS) plot of micronized rifampicin. FIG. 7C is a DSC plot of the final twin-screw blend. FIG. 7D is a DSC plot of the final low-shear blend.

FIG. 8A, FIG. 8B, FIG. 8C, FIG. 8D, and FIG. 8E provide a collection of scanning electron microscopy (SEM) images of powder blends. FIG. 8A depicts the pre-blend SEM image; FIG. 8B depicts the SEM image after two passes through the twin-screw extruder; FIG. 8C depicts the SEM image after four passes through the twin-screw extruder; FIG. 8D depicts the SEM image of the Turbula® blended powder; and FIG. 8E depicts the SEM image of micronized rifampicin.

FIG. 9A is a scattered plot of blend homogeneity of a budesonide blend for high-shear and tumble mixing plotting the coefficient of variation (CV) as a function of time. FIG. 9B is a bar graph of continuous mixing uniformity for a budesonide blend. N=8 locations for batch and continuous mixing.

FIG. 10A, FIG. 10B, and FIG. 10C provide a collection of graphs of budesonide fine particle fraction (FPF) as a function of mixing time for low-shear batch (FIG. 10A), high-shear batch (FIG. 10B), and continuous (FIG. 10C; indicated as twin-screw fine particle fraction) mixing (N=3). * Represents p<0.05.

FIG. 11 is a graph of the geometric particle size of the budesonide powder blends as measured using laser diffraction (HELOS) using the RODOS powder disperser at a pressure drop of 3 bar and rotor speed set at 50%.

FIG. 12 is a diagram of exemplary feeding configurations for a budesonide mixture prepared by continuous mixing.

FIG. 13 illustrates the allowed operating window based on mixer and feeder limits of screw speed and feed rate.

FIG. 14A, FIG. 14B, and FIG. 14C show three plots of % recovery as a function of mixing time for twin screw mixing (FIG. 14A), low-shear batch mixing (FIG. 14B), and high-shear batch mixing (FIG. 14C).

FIG. 15A, and FIG. 15 B shows two plots and FIG. 15C shows a bar graph illustrating budesonide fine particle fraction (FPF) as a function of mixing time for low-shear batch (FIG. 15A), high-shear batch (FIG. 15B), and continuous mixing, where n=3 (FIG. 15C). * Represents p<0.05.

FIG. 16A, FIG. 16B, FIG. 16C, and FIG. 16D provide a collection of scanning electron microscopy (SEM) images of powder blends. FIG. 16A depicts a low-shear batch final mixture; FIG. 16B depicts a high-shear batch final mixture; FIG. 16C depicts powder blend after mixing 10 g/min at 500 rpm; and FIG. 16D depicts powder blend after mixing 10 g/min at 50 rpm.

FIG. 17 is an actual by predicted plot of FPF for continuous mixing where RMSE indicates root mean square error and RSq indicates the R² value.

FIG. 18A and FIG. 18B provide a set of leverage plots of screw speed (FIG. 18A; RPM) and feed rate (FIG. 18B; g/min).

FIG. 19 illustrates screw profiles investigated using continuous mixing for conveying (top panel), 30-degree (middle panel), and 60-degree (bottom panel) screws.

FIG. 20 shows two bar graphs related to blend homogeneity of conveying (top) and 30-degree kneading profiles (bottom).

FIG. 21 shows a bar graph depicting aerosol performance of conveying and 30-degree kneading profiles.

FIG. 22 provides a collection of scanning electron microscopy (SEM) images of powder blends produced using 30-degree kneading profiles.

FIG. 23 shows a bar graph depicting aerosol performance of a powder blend passed through a twin-screw continuous mixer multiple times.

FIG. 24A, FIG. 24B, and FIG. 24C provide a collection of scanning electron microscopy (SEM) images of blend passed through twin-screw continuous mixer (FIG. 24A) 1 time, (FIG. 24B) 2 times, and (C) 3 times.

FIG. 25A provides an illustration of the general mixing profile used to prepare aerosol powders. The boxed section shows a GFM-3-15-30 element, two KB-7-3-15-30 elements, two KB-7-3-15-30-N elements. GFA X-XX-XX: conveying-trilobal-pitch length (mm)-screw length (mm). KB-Y-Y-YY-YY: kneading-trilobal-pitch length (mm)-screw length (mm). KB-Z-Z-ZZ-ZZ-N: kneading-trilobal-pitch length (mm)-screw length (mm)-neutral. FIG. 25B depicts a GFM-3-15-30 element, FIG. 25C depicts a KB-7-3-15-30 element, FIG. 25D depicts a KB-7-3-15-30-N element.

FIG. 26 shows a bar graph of content uniformity % RSD plotted as a function of screw speed and feed rate for the screw profiles containing a combing or a 30° kneading element.

FIG. 27 shows a bar graph of % FPF plotted as a function of screw speed and feed rate for the screw profiles containing a combing or a 30° kneading element (bars represent mean±standard deviation, n=3).

FIG. 28A and FIG. 28B show bar graphs summarizing the aerosol performance of powder passed through the twin-screw mixer multiple times at 40 g/min, 200 rpm using the screw profile containing the combing element. FIG. 28A shows a bar graph of % FPF for one, two, and three passes through the mixer. FIG. 28B shows a bar graph of % RSD of FPF for one, two, and three passes through the mixer.

FIG. 29A and FIG. 29B show plots of particle size fractions as a function of specific energy for the screw profiles containing a combing element (FIG. 29A) or a 30° kneading element (FIG. 29B).

FIG. 30A and FIG. 30B show overlay graphs of PSD for each specific energy condition for the screw profiles containing a combing element (FIG. 30A) and 30° kneading element (FIG. 30B).

FIG. 31 is a plot depicting span of aerosol powders plotted as a function of specific energy for the screw profiles containing a combing or a 30° kneading element.

FIG. 32A, FIG. 32B, and FIG. 32C provide a collection of scanning electron microscopy (SEM) images of 40 g/min, 200 rpm powder blends after one (FIG. 32A), two (FIG. 32B), and three (FIG. 32C) passes through the mixer using the screw profile containing a combing element. FIG. 32D is a SEM image of a 10 g/min, 500 rpm powder blend using the screw profile containing a 30° kneading element.

### DETAILED DESCRIPTION

Described herein are dry powder inhalation (DPI) mixtures for pulmonary delivery and processes for making the same. Specifically, described herein is a novel continuous manufacturing process for blending of DPI mixtures. The continuous manufacturing process described herein can provide increases in efficiency, improvements in quality, and flexibility, in addition to eliminating batch-to-batch variability. To achieve continuous powder blending, powders to be mixed enter the mixer in an uninterrupted flow, are processed until desired blend uniformity is achieved, and are discharged from the mixer, also in a continuous flow, to then be subsequently dispensed into the container closure system. The continuous flow of powder through the system is essential for an equilibrium to be maintained between the input and output from the system and ensures that the powder, regardless of time of entry, receives the same energy input and equivalent degree of mixing. Until now, continuous powder mixing has been limited to applications where large-scale powder processing requires rapid rates of production. These applications typically utilize rotating drum or ribbon blender instrumentation. Currently, these types of mixers provide limited precision and reduced flexibility and may not be capable of processing materials where strict blend uniformity is required.

The use of the multi-screw extruder as a continuous processor has been previously reported for various pharmaceutical applications including tablet processing, drying of powders, melt granulation, and others. However, several important features are unique to the case of inhalation powder blending, including (1) generally high potency of drug with majority of formulation comprising excipients (on the order of 1% w/w drug loading), (2) disparity in particle size distributions of drug (˜1-5 microns) and excipients (˜75-125 microns), and (3) the importance of the adhesion forces between the drug and excipient for the performance and lung delivery of the drug.

The present disclosure is directed to such continuous manufacturing process, and the examples provided herein demonstrate the power of the process through model drugs. Importantly, the presently disclosed multi-screw method is compared to low shear blending in a tumble mixer, which is commonly used in DPI formulation processing. Blend uniformity as a function of processing time and processing method were investigated, along with the influence of multi-screw processing time on aerosol performance. As demonstrated herein, desirable blend uniformity and aerosol performance is achieved using multi-screw processing, and surprisingly, aerosol performance is improved while simultaneously improving blend homogeneity.

### Multi-Screw Extruder

In one possible arrangement of the multi-screw extruder used herein, at least two screw bodies are housed in a barrel of its body in a manner such that the two screw bodies extend parallel to and mate with each other. Optionally, several screw bodies (e.g., two or more, three or more, four or more, five or more, etc.) are housed in a barrel in a manner such that they extend parallel to each other.

The multi-screw extruder body may be provided with a feed section having a feed port through which the extrusion material is supplied, a conveying section for conveying the material delivered through the feed section, an air vent section on the downstream side of the feed port, and an outlet section for the conveyed material, connected to the downstream side of the conveyed section. Optionally, the multi-screw extruder body may be provided with a feed section having a feed port through which the extrusion material is supplied, a kneading section for kneading the material delivered through the feed section, a conveying section for conveying the material delivered through the kneading section, an air vent section on the downstream side of the feed port, and an outlet section for the kneaded and conveyed material, connected to the downstream side of the conveying section. The air vent section may serve to discharge air contained in the extrusion material kneaded in the kneading section or conveyed in the conveying section, thereby preventing a backflow of the material. Optionally, the multi-screw extruder body may not have an air vent section. Optionally, the multi-screw extruder body may be provided with a feed section having a feed port through which the extrusion material is supplied, a kneading section for kneading the material delivered through the feed section, and a conveying section for conveying the material delivered through the kneading section. Optionally, the multi-screw extruder body may be provided with a feed section having a feed port through which the extrusion material is supplied, a kneading section for kneading the material delivered through the feed section, and a conveying section for conveying the material delivered through the kneading section. Optionally, the multi-screw extruder body may be provided with more than one feed section having more than one feed ports through which more than one extrusion material may be supplied, more than one kneading section for kneading the material delivered through the more than one feed sections, and/or more than one conveying section for conveying the material delivered through more than one kneading sections.

The multi-screw extruder may be operated in the following manner. As the multi-screw bodies are rotated (e.g., two-screw bodies), the extrusion material is delivered to the kneading section by the feed screws at the feed section. This extrusion material is kneaded by the kneading screws at the kneading section, and then continuously extruded through the outlet section. Optionally, the multi-screw bodies are rotated, the extrusion material is delivered to the kneading section by the feed screws at the feed section, the extrusion material is then kneaded by the kneading screws at the kneading section, the kneaded extrusion material is then conveyed by conveyer screws at the conveying section, and then continuously extruded through the outlet section. Optionally, the steps of feeding, kneading and conveying may be repeated before extrusion through the outlet section. Optionally, the steps are feeding and conveying. Optionally, the steps are feeding and kneading. In all iterations of the multi-screw extruder body provided herein, each screw body has a feed screw at the feed section, a kneading screw at the kneading section, and a conveying screw at the conveying section.

Where a vent is part of the multi-screw extruder body, the vent serves to remove air contained in the material being extruded. This prevents the air contained in the extrusion material from flowing back toward the feed port, so that lowering the extrusion capabilities of the extruder can be avoided.

Conventionally, 1-lobe intermeshing screws are used as the feed screws at the feed section of a multi-screw extruder (e.g., twin-screw extruder). A screw flight is helically wound around the outer peripheral surface of each of the screw bodies. The screw flight of the one screw body on the upstream side of the feed section is engagedly interposed between each two adjacent turns of a screw flight of the other screw body. Optionally, 2-lobe intermeshing screws are used as the feed screws at the feed section of the multi-screw extruder. Optionally, 3-lobe intermeshing screws are used as the feed screws at the feed section of the multi-screw extruder. Optionally, other multi-lobe intermeshing screws are used as the feed screws at the feed section of the multi-screw extruder.

The multi-screw extruder may further have one or more of a melt and mix section and/or a mix and seal section following the feed section. Optionally, the multi-screw extruder my only have a melt and mix section. Optionally, the multi-screw extruder may only have a mix and seal section. The multi-screw extruder may facilitate the application of moisture. The multi-screw extruder may be amenable to high temperatures (e.g., temperatures up to about 200° C.). In some cases, the temperature of one or more sections of the multi-screw extruder can be controlled to be 200° C. or less (e.g., 190° C. or less, 180° C. or less, 170° C. or less, 160° C. or less, 150° C. or less, 140° C. or less, 130° C. or less, 120° C. or less, 110° C. or less, or 100° C. or less). For example, the temperature of one or more sections of the multi-screw extruder can be controlled to be from 40° C. to 200° C. The multi-screw extruder may further remove static following the feed section, the kneading section, or the conveying section.

### Dry Powder Inhaler Blend Processing

Provided herein is a process for making a dry powder inhaler blend. The process includes introducing an active pharmaceutical ingredient, a lubricant, and a carrier into a multi-screw extruder and continuously mixing the active pharmaceutical ingredient, the lubricant, and the carrier in the multi-screw extruder to form a dry powder inhaler blend. In this method, the active pharmaceutical ingredient, the lubricant, and the carrier are in the form of a powder.

The multi-screw extruder for use in the process described herein includes at least two co-rotating screws and an intermeshing region, which is positioned between the at least two co-rotating screws. A suitable multi-screw extruder for use in the present process includes, for example, a Leistritz Nano-16 co-rotating multi-screw extruder (American Leistritz Extruder Corp., Somerville, NJ). Optionally, the screw profile can include primarily conveying elements and one grooved mixing (GFM) element towards the end of the screw.

Scaling factors depend on feed capacity (e.g., volumetric scale-up), motor power (e.g., power scale-up), and heat input (e.g., heat transfer scale-up). Volumetric scale-up of the extrusion process focuses on maintaining a constant mean residence time. The volumetric scale-up strategy applies when free volume limits the throughput, or where the extruder is operating at its volumetric limits. This can occur, for example, when the feed zone is full as a result of low material density and/or conveying capacity. In such a case, increasing the screw speed increases the throughput. Power scale-up is based on constant specific mechanical energy (SME). As used herein, constant SME refers to constant energy per unit mass. Maintaining constant SME during scaling can be critical, because the energy input can be essential to achieve the desired product. Finally, the heat transfer scale-up strategy can be adopted when the extrusion process is limited by heat transfer, and the desired melt temperature may not be achieved.

Optionally, the multi-screw extruder for use in the methods described herein can be equipped with a volumetric feeder to control the feed rate of components into the extruder. The feed rate for introducing the components into the multi-screw extruder can be controlled at, for example, 1 g/min to 50 g/min. Optionally, the feed rate can be 2 g/min to 40 g/min, 3 g/min to 35 g/min, 4 g/min to 30 g/min, or 5 g/min to 25 g/min.

The multi-screw extruder can be operated at any suitable speed for ensuring the desired mixing is accomplished in the desired time frame. In some examples, the multi-screw extruder is operated at a screw speed of rotation of at least 50 rpm. For example, the screw speed of rotation can be at least 50 rpm. In some cases, the screw speed of rotation can be from 50 rpm to 1000 rpm (e.g., from 100 rpm to 900 rpm, from 200 rpm to 800 rpm, or from 300 rpm to 700 rpm).

The components for use in the process described herein can include at least one active pharmaceutical ingredient, at least one lubricant, and a carrier. Each of the components is in the form of a powder.

### Active Pharmaceutical Ingredient

A suitable active pharmaceutical ingredient (also referred to herein as “API”) for use in the methods described herein includes any API known to those of skill in the art. In some examples, the API for use in the methods described herein are agents that are suitable for pulmonary delivery or administration by inhalation. In some cases, the API can include agents that are suitable for treating a pulmonary disease or pulmonary infection. Suitable APIs include, for example, antiallergics, anticancer agents, antifungals, antineoplastic agents, analgesics, bronchodilators, antihistamines, antiviral agents, antitussives, anginal preparations, antibiotics, anti-inflammatories, immunomodulators, 5-lipoxygenase inhibitors, leukotriene antagonists, phospholipase A2 inhibitors, phosphodiesterase IV inhibitors, peptides, proteins, steroids, and vaccine preparations. In some examples, the API can be selected from adrenaline, albuterol, atropine, beclomethasone dipropionate, budesonide, butixocort propionate, clemastine, cromolyn, epinephrine, ephedrine, fentanyl, flunisolide, fluticasone, formoterol, ipratropium bromide, isoproterenol, lidocaine, morphine, nedocromil, pentamidine isoethionate, pirbuterol, prednisolone, rifampicin, salmeterol, terbutaline, tetracycline, and pharmaceutically acceptable salts and solvates thereof, and mixtures thereof. In some cases, the API for use in the methods described herein is a low dose API. As used herein, low dose API refers to an API that make-up less than 5% of the composition. For example, for a 400 mg dosage form, a low dose API would make up less than 20 mg of the composition. In some cases, the API for use in the methods described herein is a potent oral drug. As used herein, a potent oral drug is a drug having a biological activity at less than or equal to 150 μg per kg body mass in humans (which can be equivalent to a therapeutic dose of less than or equal to 10 mg).

The active pharmaceutical ingredient can be present in blend resulting from the method in an amount of 5 wt. % or less (e.g., 4 wt. % or less, 3 wt. % or less, 2 wt. % or less, 1 wt. % of less, or 0.5 wt. % or less). For example, the active pharmaceutical ingredient can be present in the blend in an amount of 0.01 wt. % to 5 wt. %, 0.1 wt. % to 4.5 wt. %, 0.2 wt. % to 4.0 wt. %, 0.5 wt. % to 3.5 wt. %, or 1 wt. % to 3 wt. %. In some cases, the active pharmaceutical ingredient is present in the blend in an amount of 0.01 wt. %, 0.05 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, or 5.0 wt. %.

The geometric particle size of the components of the blends, and of the blends themselves (as further detailed below), can be measured using laser diffraction with measurements performed using, for example, a RODOS powder disperser. By way of example, the measurements can be performed by controlling the disperser settings to a pressure drop of 3 bar and a rotor speed set at 50%, though these variables can be adjusted as desired by one of ordinary skill in the art given the guidance provided herein. Measurements with an optical concentration in a certain range, such as between 5% and 25%, can be considered for analysis. The particles diameter can be expressed as the mean Xₙ diameter values, where n % of the particles have a diameter <X (e.g., X₁₀, X₅₀, X₉₀). In other words, the X₁₀ value means that 10% of the measured particles have a diameter that is less than or equal to the value. Similarly, the X₅₀ value means that 50% of the measured particles have a diameter that is less than or equal to the indicated value, and the X₉₀ value means that 90% of the measured particles have a diameter that is less than or equal to the indicated value. A measure of particle size distribution breadth can be represented by the calculated factor “Span,” according to the following calculation:

(X₉₀−X₁₀)/X₅₀.

In some cases, the active pharmaceutical ingredient for use in the blend has an X₉₀ value, prior to being introduced into the extruder, of 10 μm or less. For example, the X₉₀ value of the active pharmaceutical ingredient can be from 5 μm to 10 μm, from 5.5 μm to 9 μm, or from 6 μm to 8 μm. Optionally, the span for the active pharmaceutical agent for use in the blend can be from 0.1 μm to 8 μm, from 0.5 μm to 6 μm, or from 1 μm to 4 μm.

In some cases, the dry powder inhaler blend has a coefficient of variation percent (% CV) value of less than 5% for any combination of screw speed and feed rate. As used herein, coefficient of variation percent (% CV) is defined as a statistical measure of the relative dispersion of data points in a data series around a mean. Herein, a low % CV value signifies a low percent of dispersion among data points for the dry powder inhaler blend using the multi-screw extrusion method with any combination of screw speed and feed rate tested, indicating good reproducibility. In some cases, the % CV value is less than 5% where the screw speed is 50 rpm and the feed rate is 10 g/min. In some cases, the % CV value is less than 5% where the screw speed is 100 rpm and the feed rate is 20 g/min. In some cases, the % CV value is less than 5% where the screw speed is 200 rpm and the feed rate is 25 g/min. In some cases, the % CV value is less than 5% where the screw speed is 200 rpm and the feed rate is 40 g/min. In some cases, the % CV value is less than 5% where the screw speed is 300 rpm and the feed rate is 10 g/min. In some cases, the % CV value is less than 5% where the screw speed is 350 rpm and the feed rate is 40 g/min. In some cases, the % CV value is less than 5% where the screw speed is 500 rpm and the feed rate is 10 g/min. In some cases, the % CV value is less than 5% where the screw speed is 500 rpm and the feed rate is 40 g/min.

### Lubricant

As noted above, the powder blend further includes one or more lubricants. The lubricants can be useful, for example, for achieving continuous powder conveyance through compartments of the extruder. The lubricant can reduce the adhesion between the carrier and the surfaces of the extruder, thus avoiding the build-up of powder within the extruder. Suitable lubricants for use as a component in the methods described herein include any suitable typically used in powder formulations for active pharmaceutical ingredients. In some examples, the lubricant is a stearate, such as magnesium stearate, calcium stearate, and/or sodium stearate. In some examples, the lubricant is glycerin monostearate, glyceryl behenate, blyceryl palmitostearate, hydrogenated castor oil, hydrogenated vegetable oil type I, light mineral oil, magnesium lauryl sulfate, medium-chain triglycerides, mineral oil, myristic acid, palmitic acid, poloxamer, polyethylene glycol, sodium benzoate, sodium chloride, sodium lauryl sulfate, sodium stearyl fumarate, stearic acid, talc, and/or zinc stearate.

The lubricant can be present in blend resulting from the method in an amount of 1 wt. % or less (e.g., 0.8 wt. % or less, 0.7 wt. % or less, 0.6 wt. % or less, 0.5 wt. % of less, or 0.5 wt. % or less). For example, the lubricant can be present in the blend in an amount of 0.01 wt. % to 1 wt. %, 0.1 wt. % to 0.9 wt. %, 0.2 wt. % to 0.8 wt. %, 0.3 wt. % to 0.7 wt. %, or 0.35 wt. % to 0.6 wt. %. In some cases, the lubricant is present in the blend in an amount of 0.01 wt. %, 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.6 wt. %, 0.65 wt. %, 0.7 wt. %, 0.75 wt. %, 0.8 wt. %, 0.85 wt. %, 0.9 wt. %, 0.95 wt. %, or 1.0 wt. %.

In some cases, the lubricant for use in the blend has an X₉₀ value, prior to being introduced into the extruder, of 20 μm or less. For example, the X₉₀ value of the lubricant can be from 5 μm to 20 μm, from 10 μm to 19 μm, or from 12 μm to 18 μm. Optionally, the span for the lubricant for use in the blend can be from 0.1 m to 8 μm, from 0.5 μm to 6 μm, or from 1 μm to 4 μm.

### Carrier

The powder blend can further include one or more carriers. The carrier can be any pharmacologically inert material that can be used for inhalation. Suitable carriers for use in the powder blends described herein can be, for example, a sugar alcohol or a polyol. In some examples, the carrier is or includes lactose (e.g., inhalation grade lactose), ammonium alginate, calcium carbonate, calcium lactate, calcium phosphate, dibasic anhydrous, dibasic dehydrate, tribasic, calcium silicate, calcium sulfate, cellulose powdered, silicified microcrystalline, cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, anhydrous, monohydrate and corn starch, monohydrate and microcrystalline cellulose, spray dried, magnesium carbonate, magnesium oxide, maltodextrin, and/or maltose. The carrier can account for the remainder of the weight percentage of the powder blend. In some examples, the carrier can be present in an amount ranging from 94 wt. % to 99.5 wt. % of the blend.

The carrier can have a large particle size relative to the other components of the powder blend, thus allowing lubricant and active pharmaceutical ingredient particles to adhere to the surfaces of the carrier particle. The carrier is appropriately selected to allow for the release of the active pharmaceutical ingredient when administered to the patient (e.g., within the lungs of the patient). The carrier can optionally be processed through a sieve of the appropriate size to control carrier size to the processed in the methods described herein.

In some cases, the lubricant for use in the blend has an X90 value, prior to being introduced into the extruder, of 80 μm or greater. For example, the X₉₀ value of the lubricant can be from 80 μm to 200 μm, from 90 μm to 190 μm, or from 100 μm to 150 μm. Optionally, the span for the lubricant for use in the blend can be from 20 μm to 75 μm, from 22.5 μm to 70 μm, or from 25 μm to 65 μm.

### Pre-Blends and Blending

Optionally, the lubricant and carrier can be combined to form a pre-blend prior to introducing the components into the extruder or a feeder attached to the extruder. The pre-blend can be prepared using any suitable blending or mixing apparatus, such as a tumble blender or V-blender. The lubricant and/or carrier can be filtered using, for example, a sieve to control the particle size of the components for introduction into the extruder.

In some cases, the active pharmaceutical ingredient can also be included in a pre-blend with one or more of the other components prior to introducing the components into the extruder or feeder attached to the extruder.

In some cases, no pre-blending is performed and each component is separately introduced into the extruder using one or more feeders.

Once introduced into the extruder, either as separate components through one or more feeders or as optional pre-blends, the components can be continuously mixed in the multi-screw extruder as further described in the examples below. The processing time can be varied to achieve the desired mixing. In some cases, the processing time can be varied by performing additional cycles, e.g., by feeding the mixed blend back through the extruder one or more additional times.

The dry powder inhaler blend, after mixing according to the multi-screw extruder methods described herein, can have a finer particle size distribution than blends prepared using other methods, such as low-shear or high-shear mixers. In some examples, the dry powder inhaler blend prepared according to the methods described herein can have an X₉₀ particle size lower than the X₉₀ particle size of a dry powder inhaler blend prepared using low-shear or high-shear mixing. In some cases, the X₉₀ particle size is at least 10% lower than the X₉₀ particle size of the dry powder inhaler blend prepared using low-shear or high-shear mixing. For example, the X₉₀ particle size can be at least 15% lower, at least 20% lower, at least 25% lower, at least 30% lower, at least 35% lower, at least 40% lower, at least 45% lower, or at least 50% lower than the X₉₀ particle size of the same dry powder inhaler blend (e.g., a blend having the same components as used for the blend prepared according to the methods described herein) prepared using low-shear or high-shear mixing.

In some cases, the dry powder inhaler blend has a coefficient of variation percent (% CV) value of less than 5% for any combination of screw speed and feed rate. Herein, a low % CV value signifies a low percent of dispersion among data point for the dry powder inhaler blend using the multi-screw extrusion method with any combination of screw speed and feed rate tested which implies high reproducibility. In some cases, the % CV value is less than 5% where the screw speed is 50 rpm and the feed rate is 10 g/min. In some cases, the % CV value is less than 5% where the screw speed is 100 rpm and the feed rate is 20 g/min. In some cases, the % CV value is less than 5% where the screw speed is 200 rpm and the feed rate is 25 g/min. In some cases, the % CV value is less than 5% where the screw speed is 200 rpm and the feed rate is 40 g/min. In some cases, the % CV value is less than 5% where the screw speed is 300 rpm and the feed rate is 10 g/min. In some cases, the % CV value is less than 5% where the screw speed is 350 rpm and the feed rate is 40 g/min. In some cases, the % CV value is less than 5% where the screw speed is 500 rpm and the feed rate is 10 g/min. In some cases, the % CV value is less than 5% where the screw speed is 500 rpm and the feed rate is 40 g/min.

As described in the examples below, the dry powder inhaler blends can have improved drug potency and powder uniformity after processing using a multi-screw extruder, as compared to blends processed using low-shear mixing. In addition, the drug potency and powder uniformity can be further enhanced by increasing the mixing time and/or mixing cycles within the multi-screw extruder without compromising aerosol performance (see Example 1).

### Exemplary Dry Powder Inhaler Blends

Exemplary blends for use as the dry powder inhaler blends and can include rifampicin as the active pharmaceutical ingredient in an amount of 5 wt. % or less, a lubricant (e.g., magnesium stearate) in an amount of 1 wt. % or less, and a carrier (e.g., lactose). The X₉₀ particle size of the rifampicin dry powder inhaler blend can be, for example, less than 6.5 μm after processing (e.g., continuous mixing in a multi-screw extruder) according to the methods described herein. Optionally, the X₉₀ particle size of the rifampicin dry powder inhaler blend can be, for example, less than 6.45 μm.

In some cases, the dry powder inhaler blend including rifampicin as the active pharmaceutical ingredient has a % CV value of less than 5% for any combination of screw speed and feed rate. In some cases, the % CV value is less than 5% where the screw speed is 50 rpm and the feed rate is 10 g/min. In some cases, the % CV value is less than 5% where the screw speed is 100 rpm and the feed rate is 20 g/min. In some cases, the % CV value is less than 5% where the screw speed is 200 rpm and the feed rate is 25 g/min. In some cases, the % CV value is less than 5% where the screw speed is 200 rpm and the feed rate is 40 g/min. In some cases, the % CV value is less than 5% where the screw speed is 300 rpm and the feed rate is 10 g/min. In some cases, the % CV value is less than 5% where the screw speed is 350 rpm and the feed rate is 40 g/min. In some cases, the % CV value is less than 5% where the screw speed is 500 rpm and the feed rate is 10 g/min. In some cases, the % CV value is less than 5% where the screw speed is 500 rpm and the feed rate is 40 g/min.

Additional exemplary blends for use as the dry powder inhaler blends and can include budesonide as the active pharmaceutical ingredient in an amount of 5 wt. % or less, a lubricant (e.g., magnesium stearate) in an amount of 1 wt. % or less, and a carrier (e.g., lactose). The X₉₀ particle size of the budesonide dry powder inhaler blend can be, for example, less than 6 μm after processing (e.g., continuous mixing in a twin-screw extruder) according to the methods described herein. Optionally, the X₉₀ particle size of the budesonide dry powder inhaler blend can be, for example, less than 5.90 μm.

In some cases, the dry powder inhaler blend including budesonide as the active pharmaceutical ingredient has a % CV value of less than 5% for any combination of screw speed and feed rate. In some cases, the % CV value is less than 5% where the screw speed is 50 rpm and the feed rate is 10 g/min. In some cases, the % CV value is less than 5% where the screw speed is 100 rpm and the feed rate is 20 g/min. In some cases, the % CV value is less than 5% where the screw speed is 200 rpm and the feed rate is 25 g/min. In some cases, the % CV value is less than 5% where the screw speed is 200 rpm and the feed rate is 40 g/min. In some cases, the % CV value is less than 5% where the screw speed is 300 rpm and the feed rate is 10 g/min. In some cases, the % CV value is less than 5% where the screw speed is 350 rpm and the feed rate is 40 g/min. In some cases, the % CV value is less than 5% where the screw speed is 500 rpm and the feed rate is 10 g/min. In some cases, the % CV value is less than 5% where the screw speed is 500 rpm and the feed rate is 40 g/min.

### Dry Powder Inhaler Blend Capsules and Methods of Use

Also described herein are capsules for use in inhalers. The capsules can include a dry powder inhaler blend as described herein. In some cases, the formulations described herein can be used for the treatment for lung, digestive, hepatic, and biliary tract related diseases and disorders. The formulations as described herein, with or without additional agents, can be provided in the form of an inhaler or nebulizer for inhalation therapy. As used herein, inhalation therapy refers to the delivery of a therapeutic agent, such as the compounds described herein, in an aerosol form to the respiratory tract (e.g., pulmonary delivery). As used herein, the term aerosol refers to very fine solid particles delivered under pressure to a site of therapeutic application. In some examples, the term aerosol refers to very fine powder or solid particles carried by a propellant gas under pressure to a site of therapeutic application. When a pharmaceutical aerosol is employed, the aerosol contains the one or more compounds described herein. The aerosol can be in the form of a powder or semi-solid preparation, for example. In the case of a powder, no propellant gas is required when the device is a breath activated dry powder inhaler. In some examples, aerosols employed are intended for administration as fine, solid particles or as liquid mists via the respiratory tract of a subject. Optionally, the propellant of an aerosol package containing one or more compounds described herein can be capable of developing sufficient pressure to expel the compound when a valve on the aerosol package is opened. Without limitation, various types of propellants can be utilized, such as fluorinated hydrocarbons (e.g., trichloromonofluromethane, dichlorodifluoromethane, and dichlorotetrafluoroethane) and compressed gases (e.g., nitrogen, carbon dioxide, nitrous oxide, or Freon). As described herein, the aerosol can be in the form of a powder; thus, no propellant gas is required when the device is a breath activated dry powder inhaler. Aerosols employed are intended for administration as fine, solid particles via the respiratory tract of a patient.

### Low Dose Dry Powder Blend Processing

Provided herein is a process for making a low dose dry powder blend. In this process, the active pharmaceutical ingredient, the lubricant, and the carrier are in the form of a powder. The process includes introducing an active pharmaceutical ingredient, a lubricant, and a carrier into a multi-screw extruder and continuously mixing the active pharmaceutical ingredient, the lubricant, and the carrier in the multi-screw extruder to form a dry powder blend wherein less than 5% of the composition is the active pharmaceutical ingredient. Optionally, less than 4% of the composition is the active pharmaceutical ingredient. Optionally, less than 3% of the composition is the active pharmaceutical ingredient. Optionally, less than 2% of the composition is the active pharmaceutical ingredient. Optionally, less than 1% of the composition of the active pharmaceutical ingredient. As used herein, a low dose dry powder blend refers to a dry powder blend wherein less than 5 wt. % (e.g., less than 4 wt. %, less than 3 wt. %, less than 2 wt. %, less than 1 wt. %, or less than 0.5 wt. %) of the composition is the active pharmaceutical ingredient.

The multi-screw extruder for use in the process described herein includes at least two co-rotating screws and an intermeshing region, which is positioned between the at least two co-rotating screws as described for the “Dry Powder Inhaler Blend”.

### Active Pharmaceutical Ingredient

A suitable active pharmaceutical ingredient (also referred to herein as “API”) for use in the methods described herein includes any API known to those of skill in the art. In some examples, the API for use in the methods described herein are agents that are suitable for administration by mouth. For example, low dose APIs refers to APIs that make-up less than 5 wt. % of the composition (e.g., less than 4 wt. %, less than 3 wt. %, less than 2 wt. %, less than 1 wt. %, or less than 0.5 wt. % of the composition).

A low dose API may be incorporated in a dosage form comprising a dry powder blend comprising the API, a lubricant, and a carrier, wherein the dry powder blend is prepared according to the process for making a “dry powder inhaler blend.” As used herein, a dosage form may be a tablet, a capsule, a paracrystalline powder, a gel or a liquid. The dosage form may be administered through oral, buccal, sublingual, rectal, intravenous, intra-arterial, intraosseous, intramuscular, intracerebral, intraventricular, or intrathecal administration. Administration may further be subcutaneous administration, intraperitoneal administration, intraocular administration, intranasal administration, transdermal administration, epidural administration, intracranial administration, transdermal administration, intravaginal administration, intrauterine administration, intravitreal administration, or transmucosal administration. Optionally, the dosage form may be administered by injection. In some cases, the API further comprises an antibacterial agent or a steroid.

The active pharmaceutical ingredient (“API”) can be present in blend resulting from the method in an amount of 5 wt. % or less (e.g., 4 wt. % or less, 3 wt. % or less, 2 wt. % or less, 1 wt. % of less, or 0.5 wt. % or less). For example, the active pharmaceutical ingredient can be present in the blend in an amount of 0.01 wt. % to 5 wt. %, 0.1 wt. % to 4.5 wt. %, 0.2 wt. % to 4.0 wt. %, 0.5 wt. % to 3.5 wt. %, or 1 wt. % to 3 wt. %. In some cases, the active pharmaceutical ingredient is present in the blend in an amount of 0.01 wt. %, 0.05 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, or 5.0 wt. %.

### Lubricant

As noted above, the powder blend further includes one or more lubricants. The lubricants can be useful, for example, for achieving continuous powder conveyance through compartments of the extruder. The lubricant can reduce the adhesion between the carrier and the surfaces of the extruder, thus avoiding the build-up of powder within the extruder. Suitable lubricants for use as a component in the methods described herein include any suitable typically used in powder formulations for active pharmaceutical ingredients. In some examples, the lubricant is a stearate, such as magnesium stearate, calcium stearate, and/or sodium stearate. In some examples, the lubricant is glycerin monostearate, glyceryl behenate, blyceryl palmitostearate, hydrogenated castor oil, hydrogenated vegetable oil type I, light mineral oil, magnesium lauryl sulfate, medium-chain triglycerides, mineral oil, myristic acid, palmitic acid, poloxamer, polyethylene glycol, sodium benzoate, sodium chloride, sodium lauryl sulfate, sodium stearyl fumarate, stearic acid, talc, and/or zinc stearate.

The lubricant can be present in blend resulting from the method in an amount of 1 wt. % or less (e.g., 0.8 wt. % or less, 0.7 wt. % or less, 0.6 wt. % or less, 0.5 wt. % of less, or 0.5 wt. % or less). For example, the lubricant can be present in the blend in an amount of 0.01 wt. % to 1 wt. %, 0.1 wt. % to 0.9 wt. %, 0.2 wt. % to 0.8 wt. %, 0.3 wt. % to 0.7 wt. %, or 0.35 wt. % to 0.6 wt. %. In some cases, the lubricant is present in the blend in an amount of 0.01 wt. %, 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.6 wt. %, 0.65 wt. %, 0.7 wt. %, 0.75 wt. %, 0.8 wt. %, 0.85 wt. %, 0.9 wt. %, 0.95 wt. %, or 1.0 wt. %.

In some cases, the lubricant for use in the blend has an X₉₀ value, prior to being introduced into the extruder, of 20 μm or less. For example, the X₉₀ value of the lubricant can be from 5 μm to 20 μm, from 10 μm to 19 μm, or from 12 μm to 18 μm. Optionally, the span for the lubricant for use in the blend can be from 0.1 μm to 8 μm, from 0.5 μm to 6 μm, or from 1 μm to 4 μm.

In some cases, the dry powder inhaler blend has a % CV value of less than 5% for any combination of screw speed and feed rate. In some cases, the % CV value is less than 5% where the screw speed is 50 rpm and the feed rate is 10 g/min. In some cases, the % CV value is less than 5% where the screw speed is 100 rpm and the feed rate is 20 g/min. In some cases, the % CV value is less than 5% where the screw speed is 200 rpm and the feed rate is 25 g/min. In some cases, the % CV value is less than 5% where the screw speed is 200 rpm and the feed rate is 40 g/min. In some cases, the % CV value is less than 5% where the screw speed is 300 rpm and the feed rate is 10 g/min. In some cases, the % CV value is less than 5% where the screw speed is 350 rpm and the feed rate is 40 g/min. In some cases, the % CV value is less than 5% where the screw speed is 500 rpm and the feed rate is 10 g/min. In some cases, the % CV value is less than 5% where the screw speed is 500 rpm and the feed rate is 40 g/min.

### Carrier

The powder blend can further include one or more carriers. Suitable carriers for use in the powder blends described herein can be, for example, a sugar alcohol or a polyol. In some examples, the carrier is or includes lactose (e.g., inhalation grade lactose), ammonium alginate, calcium carbonate, calcium lactate, calcium phosphate, dibasic anhydrous, dibasic dehydrate, tribasic, calcium silicate, calcium sulfate, cellulose powdered, silicified microcrystalline, cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, anhydrous, monohydrate and corn starch, monohydrate and microcrystalline cellulose, spray dried, magnesium carbonate, magnesium oxide, maltodextrin, maltose. The carrier can account for the remainder of the weight percentage of the powder blend. In some examples, the carrier can be present in an amount ranging from 94 wt. % to 99.5 wt. % of the blend.

The carrier can have a large particle size relative to the other components of the powder blend, thus allowing lubricant and active pharmaceutical ingredient particles to adhere to the surfaces of the carrier particle. The carrier is appropriately selected to allow for the release of the active pharmaceutical ingredient when administered to the patient (e.g., within the lungs of the patient). The carrier can optionally be processed through a sieve of the appropriate size to control carrier size to the processed in the methods described herein.

In some cases, the lubricant for use in the blend has an X₉₀ value, prior to being introduced into the extruder, of 80 μm or greater. For example, the X₉₀ value of the lubricant can be from 80 μm to 200 μm, from 90 μm to 190 μm, or from 100 μm to 150 μm. Optionally, the span for the lubricant for use in the blend can be from 20 μm to 75 μm, from 22.5 μm to 70 μm, or from 25 μm to 65 μm.

In some cases, the dry powder blend has a coefficient of variation percent (% CV) value of less than 5% for any combination of screw speed and feed rate. As used herein, coefficient of variation (CV) is defined as a statistical measure of the relative dispersion of data points in a data series around a mean. Herein, a low % CV value signifies a low percent of dispersion among data point for the dry powder blend using the multi-screw extrusion method with any combination of screw speed and feed rate tested implying good reproducibility. In some cases, the % CV value is less than 5% where the screw speed is 50 rpm and the feed rate is 10 g/min. In some cases, the % CV value is less than 5% where the screw speed is 100 rpm and the feed rate is 20 g/min. In some cases, the % CV value is less than 5% where the screw speed is 200 rpm and the feed rate is 25 g/min. In some cases, the % CV value is less than 5% where the screw speed is 200 rpm and the feed rate is 40 g/min. In some cases, the % CV value is less than 5% where the screw speed is 300 rpm and the feed rate is 10 g/min. In some cases, the % CV value is less than 5% where the screw speed is 350 rpm and the feed rate is 40 g/min. In some cases, the % CV value is less than 5% where the screw speed is 500 rpm and the feed rate is 10 g/min. In some cases, the % CV value is less than 5% where the screw speed is 500 rpm and the feed rate is 40 g/min.

### Pre-Blends and Blending

Optionally, the lubricant and carrier can be combined to form a pre-blend prior to introducing the components into the extruder or a feeder attached to the extruder. The pre-blend can be prepared using any suitable blending or mixing apparatus, such as a tumble blender or V-blender. The lubricant and/or carrier can be filtered using, for example, a sieve to control the particle size of the components for introduction into the extruder.

In some cases, the active pharmaceutical ingredient can also be included in a pre-blend with one or more of the other components prior to introducing the components into the extruder or feeder attached to the extruder.

In some cases, no pre-blending is performed and each component is separately introduced into the extruder using one or more feeders.

Once introduced into the extruder, either as separate components through one or more feeders or as optional pre-blends, the components can be continuously mixed in the multi-screw extruder as further described in the examples below. The processing time can be varied to achieve the desired mixing. In some cases, the processing time can be varied by performing additional cycles, e.g., by feeding the mixed blend back through the extruder one or more additional times.

As described in the examples below, the dry powder blends can have improved drug distribution after processing using a multi-screw extruder, as compared to blends processed using low-shear mixing. In addition, the drug potency and powder uniformity can be further enhanced by increasing the mixing time and/or mixing cycles within the multi-screw extruder without compromising performance (see Example 3).

Throughout this application, various publications are referenced. The disclosures of these publications in their entireties are hereby incorporated by reference into this application.

The examples below are intended to further illustrate certain aspects of the methods and compositions described herein and are not intended to limit the scope of the claims.

### EXAMPLES

### Example 1: Rifampicin Formulation Preparation

### Pre-Blend Preparation

A pre-blend of inhalation-grade lactose and magnesium stearate was prepared prior to twin-screw blending and low shear blending. A commercial inhalation-grade lactose. Lactohale 206, was used as the example inhalation grade lactose system for all studies (DFE Pharma, Klever Strasse 187, Goch, Germany). For the pre-blend, lactose was sieved through a #30 sieve, then blended with magnesium stearate (0.4% w/w) using a V-blender 25 rpm for 15 minutes. A 200 g pre-blend was prepared. As is typically required prior to batch blending, geometric dilution of the drug with the carrier system was also conducted. Adams, W. P., et al., Effects of Device and Formulation on In Vitro Performance of Dry Powder Inhalers. AAPS J. 2012, 14 (3), 400-409. Specifically, rifampicin was added by geometric dilution to create the 1% rifampicin in the carrier system. The pre-blend was assessed for initial degree of blending by sampling aliquots from three different depths across four locations in the powder bed (n=12).

Blend Preparation with Co-Rotating Twin-Screw Extruder

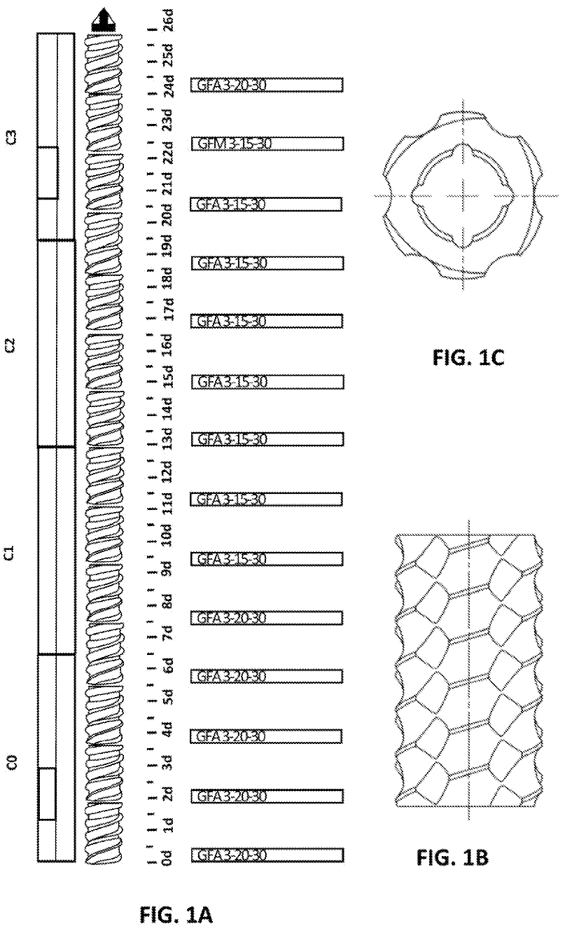

Blend preparation using a twin-screw extruder was conducted using a Leistritz Nano-16 co-rotating twin-screw extruder (American Leistritz Extruder Corp., Somerville, NJ). A twin-screw volumetric feeder (Brabender Technologies, Ontario, Canada) was used to control the feed rate at 4 g/min. The barrel of the Nano-16 extruder was divided into four zones as shown in FIG. 1A. The screw profile included primarily conveying elements and one grooved mixing (GFM) element towards the end of the screw. This profile was designed to generate appropriate press-on forces during blending. The screw speed was set at 100 rpm and steady-state torque was recorded throughout the powder processing (64 G*m). The Nano-16 extruder has minimal free volume achieved using a trilobal design of screw elements (barrel length/inner diameter of the barrel is 16 and the outer diameters of the screw elements is 15.9 mm) which helps to minimize the use of materials (<50 g). Kittikunakorn, N., et al., How Does the Dissimilarity of Screw Geometry Impact Twin-Screw Melt Granulation?Eur. J. Pharm. Sci. 2021, 157, 105645.

To assess blending as a function of processing time, the powder output from the extruder was sampled (n=3) and the powder was fed back into the extruder for additional mixing cycles (4 runs through the extruder were assessed). Each run was performed at a feed rate of 5.16 g/min.

Initial feasibility studies using pure lactose as the feed material into the twin-screw mixer showed that screw torque would gradually increase during the run and, when torque limits were reached, cause a machine shutdown to occur. Removal and inspection of the screw and barrel indicated that lactose did not flow well through the extruder and accumulated around the barrel and screw elements. Thus, to improve processing, magnesium stearate was studied for its effects on lactose powder conveyance through the instrument. In these feasibility studies, lactose/magnesium stearate mixtures were observed to have improved flow within the extruder and the powder exited the barrel in a more uniform manner. These powder flow and lubricant benefits allowed inhalation blends to be easily processed and the additional benefits of adding a ternary component into the carrier system such as improved aerosol performance improvements were also realized. Begat, P.; et al. The Role of Force Control Agents in High-Dose Dry Powder Inhaler Formulations. J. Pharm. Sci. 2009, 98 (8), 2770-2783; and Rahimpour, Y. & Hamishehkar, H. Lactose Engineering for Better Performance in Dry Powder Inhalers. Adv. Pharm. Bull. 2012, 2 (2), 183-187. Based on U.S. FDA inactive ingredients guidance and assuming a powder payload of around 20 mg, 0.4% w/w magnesium stearate lactose blends were prepared.

For example, a 200 g blend of inhalation grade lactose and magnesium stearate (0.4%) was blended using a V-blender at 25 rpm for 15 minutes. Rifampicin was air jet milled using 75 psi grind pressure, 65 psi feed pressure, and 1 g/min feed rate. Yazdi A K & Smyth H D C. Carrier-free high-dose dry powder inhaler formulation of ibuprofen: Physicochemical characterization and in vitro aerodynamic performance. Int J Pharm. 2016; 511:403-414; Yazdi A K & Smyth H D C. Hollow crystalline straws of diclofenac for high-dose and carrier-free dry powder inhaler formulations. Int J Pharm. 2016; 502:170-180; Yazdi A K & Smyth H D C. Implementation of design of experiments approach for the micronization of a drug with a high brittle-ductile transition particle diameter. Drug Dev Ind Pharm. 2017; 43:364-371. Rifampicin was added using geometric dilution to prepare the 1% pre-blend. A Leistritz Nano-16 co-rotating twin-screw extruder (American Leistritz Extruder Corp., Somerville, NJ) was used at 100 rpm in combination with a twin-screw volumetric feeder Brabender Technologies, Ontario, Canada) at 4 g/min to prepare the twin-screw blends. The screw profile included conveying elements and one GFM element to generate appropriate press-on forces.

Blend Preparation with Turbula® Mixer

Low shear blending was conducted using a Turbula® orbital mixer (Glen Mills, NJ, USA). Specifically, formulations were blended with a TurbulaR orbital mixer for a total of 40 minutes at 50 RPM. Samples were taken every 10 min up (n=3) and also assessed for blend uniformity. Donovan, M. J. & Smyth, H. D. C. Influence of Size and Surface Roughness of Large Lactose Carrier Particles in Dry Powder Inhaler Formulations. Int. J. Pharm. 2010, 402 (1), 1-9.

### Blend Uniformity and Aerosol Performance

Blend uniformity was assessed by analysis of drug content in the removed samples. Standard curves were prepared from a 1 mg/mL rifampicin stock solution prepared in 100% methanol. The stock solution was diluted in 20% ethanol to make a calibration curve with a measurable range between approximately 4 μg/mL to 125 μg/mL. Samples (10 mg of powder) were diluted in 5 mL 20% ethanol. A 1.5 mL aliquot of these solutions was removed and centrifuged at 14000 rpm for 30 minutes (to facilitate separation of drug from excipients). Drug concentration measurements were conducted using 200 μL aliquots. Ultraviolet absorbance was measured using a Tecan® Infinite® 200 PRO multimode microplate reader (Tecan Systems, Inc. San Jose, CA, USA) using Costar® Corning® 96-well UV-transparent plate at a 335 nm wavelength.

In vitro aerodynamic performance testing was conducted on blended samples at different time points and blended using different methods. Specifically, a medium resistance Plastiape RS01 dry powder inhaler device was used (Plastiape S.p.a., (Osnago, Italy). Size 3 inhalation grade hydroxypropyl methylcellulose (HPMC) capsules (Vcaps) were used (Capsugel Inc. (Morristown, New Jersey, USA)). A next generation impactor (NGI) (MSP Corporation, MN, USA) was attached sequentially to a volumetric digital flow meter (TSI 4000 Series, TSI Performance Measurement Tools, Shoreview, MN, USA), a two-way solenoid valve timer box, and a high-capacity vacuum pump (HCP5, Copley Scientific Limited, Nottingham, UK). For these studies, each HPMC capsule contained 20 mg of powder formulation. Prior to impactions, the pre-separator was loaded with 15 mL of 20% ethanol and the NGI stages were coated using a 5 mL solution of 1% (v/v) of glycerin in ethanol (subsequently evaporated). The device resistance was calculated using a dosage unit sampling apparatus according to an abbreviated Apparatus B from the USP Chapter 601 and based on the calculated device resistance, flow rate creating a 4 kPa pressure drop across the dry powder inhaler (DPI) was calculated to be approximately 60 L/m. Temperature and relative humidity were measured using an SRH77A thermo-hygrometer by Cooper-Atkins Instrument Corporation (Middlefield, CT, USA). In vitro aerodynamic performance was evaluated at the calculated flow rate for a total volume of 4 L. The inhaler and capsule were washed with 1 mL of 20% ethanol, and the 1 mL washes were stored in 1.5 mL centrifuge tubes prior to sample analysis. Drug deposition on the induction port (washed with 1 mL), pre-separator (washed with 5 mL), and stages 1-7 plus the micro-orifice collector (MOC) were each washed twice with 1 mL of ethanol and the washes were collected.

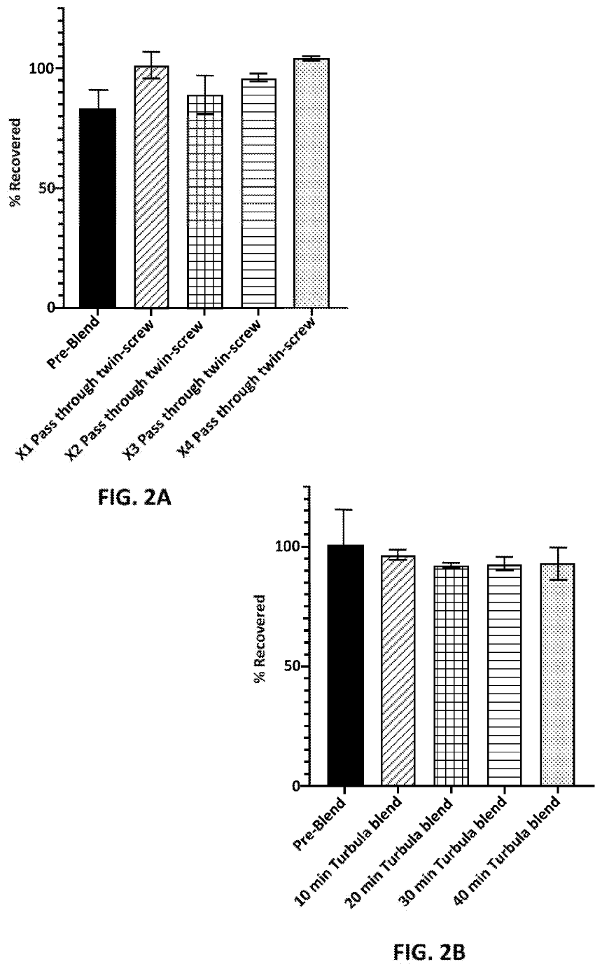

Samples from both Turbula® mixing and twin-screw mixing were analyzed for drug potency and calculated recovery and sample variability (through relative standard deviation (RSD)) were determined as shown in Table 1 and FIG. 2A and FIG. 2B.

TABLE 1
Blend uniformity of 1% rifampicin blends with 0.4% magnesium
stearate and lactose prepared by twin-screw mixing or
by Turbula ® mixing.

| Extruder Blend Uniformity | | | Turbula ® Blend Uniformity | | |
|---|---|---|---|---|---|
| | Average | | | Average | |
| | Recovered | | | Recovered | |
| | % | RSD | | % | RSD |
| Pre-Blend | 83.15 | 9.51 | Pre-Blend | 100.66 | 15.11 |
| x1 Pass through | 101.07 | 5.33 | 10 Minutes | 96.64 | 1.89 |
| twin-screw | | | | | |
| x2 Passes through | 88.88 | 8.96 | 20 Minutes | 92.43 | 0.64 |
| twin-screw | | | | | |
| x3 Passes through | 96.01 | 1.6 | 30 Minutes | 92.8 | 2.91 |
| twin-screw | | | | | |
| x4 Passes through | 104.64 | 0.24 | Final blend | 92.95 | 7.37 |
| twin-screw | | | (40 minutes) | | |

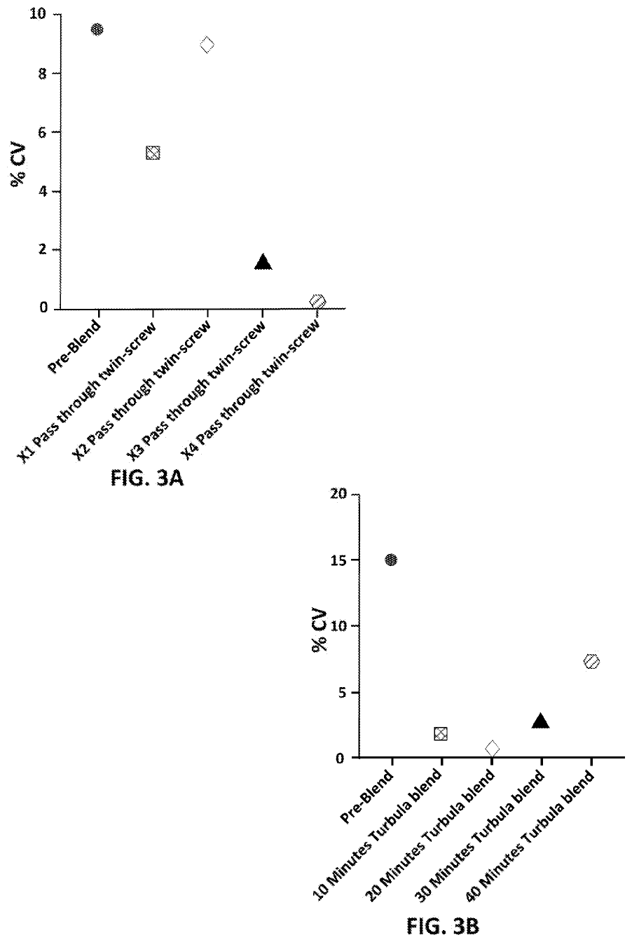

Although the Turbula® blend recovery remained high throughout on-going blending, the variance showed that the powder was dynamically mixing and de-mixing. In contrast, twin-screw mixing showed that both drug potency and powder uniformity increased with increased mixing. FIGS. 3A-3B illustrates the general decreases in variability across measures of extruder-blended DPI powder uniformity as compared to Turbula® blended powder.

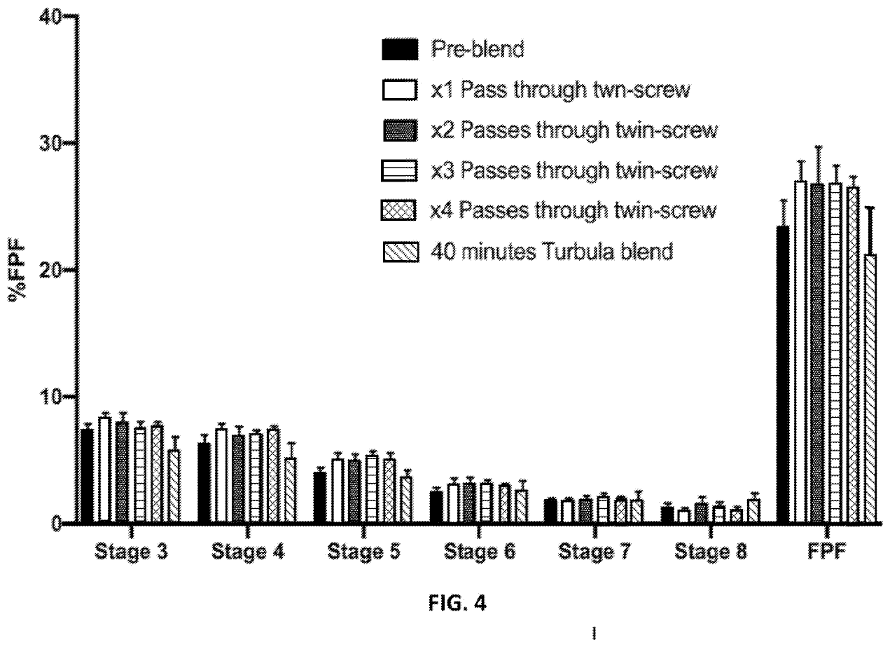

Aerosol performance was also assessed in the blends. As shown in FIG. 4, the aerosol dispersion performance of twin-screw blended powders did not decrease with increasing mixing. Moreover, the variability of the fine particle fraction as blending increase also tended to remain low relative to the pre-blended material. In comparison, the low shear Turbula® mixed blend showed similar variability in fine particle fraction. The fine particle fraction magnitude was also similar between low shear and twin-screw mixing.

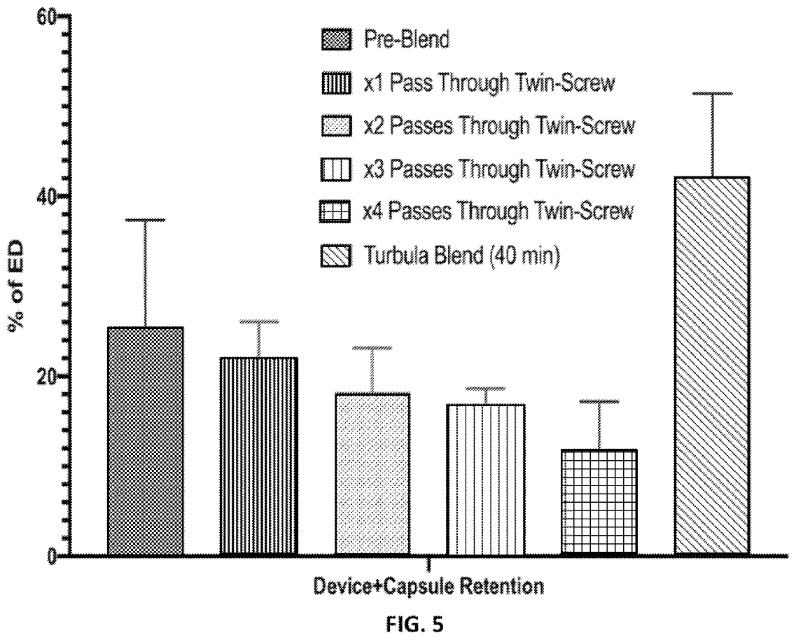

Also of note were the trends observed with drug emission from the device and capsule FIG. 5. In particular, it was observed that the amount of drug retention in the device and capsule was significantly less for the powder blends processed by twin-screw mixing compared to Turbula® blended powders. In fact, the trend was observed for increasing twin-screw mixing led to decreased device and capsule retention. Moreover, the twin-screw mixed blends had markedly less variability compared to the Turbula® blend. There were no trends discernable for the induction port or pre-separator of the NGI.

### Blend Characterization-Particle Size and Morphology

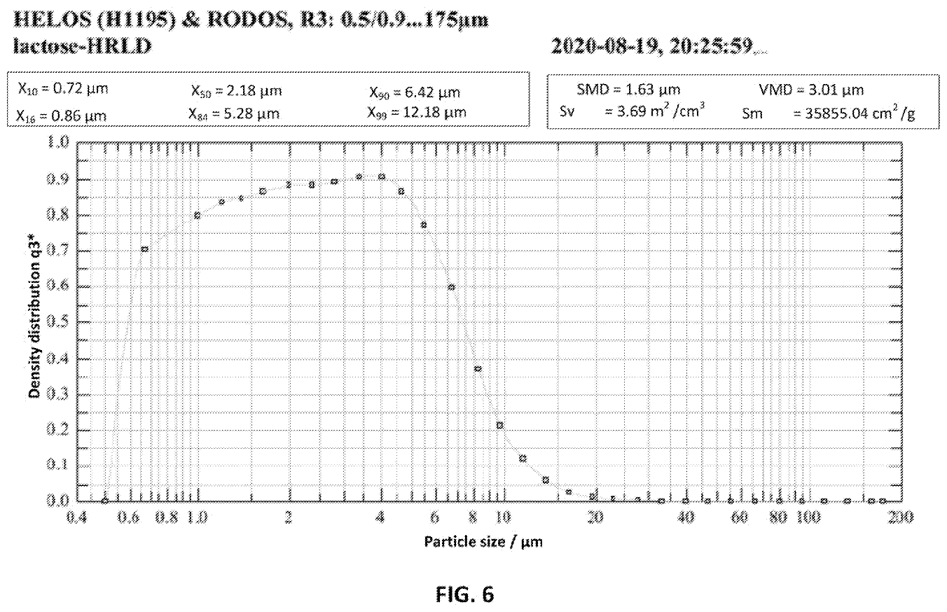

The geometric particle size of the powder blends was measured using laser diffraction (HELOS, Sympatec, Germany) for the pre-blend and after each blending cycle. Measurements were done using a RODOS powder disperser at a pressure drop of 1 bar and rotor speed set at 50% (FIG. 6). Measurements with optical concentration between 5% and 25% were considered for analysis. The particles diameter is expressed as the mean Xₙ diameter values, where n % of the particles have a diameter≤X. Jaffari, S., et al. Rapid Characterisation of the Inherent Dispersibility of Respirable Powders Using Dry Dispersion Laser Diffraction. Int. J. Pharm. 2013, 447 (1), 124-131. A measure of particle size distribution breadth is represented by the calculated factor “Span”. This was calculated using the following expression: (X₉₀−X₁₀)/X₅₀.

Results showed that the powder at various stages of blending had slightly smaller size at higher number of blending cycles. The results for particle size measurement are summarized in Table 2.

TABLE 2
RODOS dispersion measurements for 1% rifampicin
with 0.4% magnesium stearate in lactose pre-
blend and twin-screw mixed blends.

| | Particle diameter (μm) | | |
|---|---|---|---|
| | X₁₀ | X₅₀ | X₉₀ |
| Preblend | 6.8 | 54.24 | 94.49 |
| 1x Pass through twin-screw | 4.61 | 56.28 | 103.05 |
| x2 Passes through twin-screw | 3.89 | 51.78 | 100.76 |
| x3 Passes through twin-screw | 3.37 | 45.99 | 90.45 |
| X4 Passes through twin-screw | 2.27 | 38.88 | 90.6 |

Differential scanning calorimetry (DSC) was performed using aluminum sample pans and a DSC Q20 (TA Instruments, New Castle, DE). Samples were scanned up to 500° C. at a rate of 10° C./min using approximately 6 mg per sample.

Powder x-ray diffraction (PXRD) was performed using a Rigaku Miniflex 600 instrument (Rigaku Americas, The Woodlands, Texas, USA) equipped with a Cu-Kα radiation source generated at 40 kV and 15 mA. Samples were scanned in continuous mode with a step size of 0.03° over a 20 range of 4° to 45°.

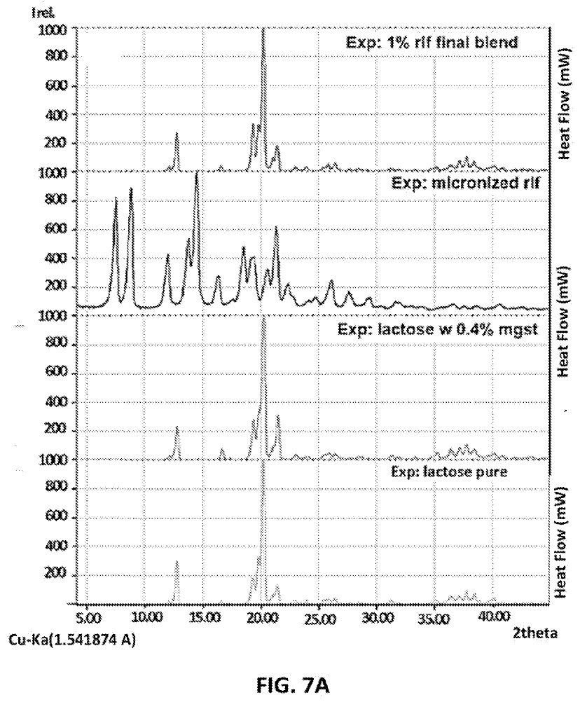

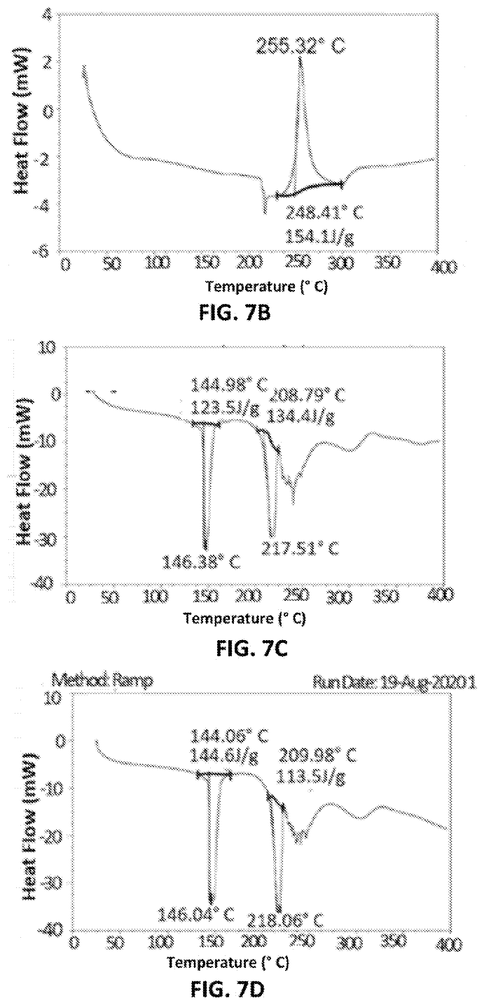

XRD analysis of the powders including the final blend from the twin-screw extruder showed no detectable changes in drug or lactose crystallinity or physical form (FIG. 7A). DSC analysis also support these observations. Rifampicin was confirmed to be Form 1 and no evidence of amorphous material was observed (see FIGS. 7B-7D). Agrawal, S., et al. Solid-State Characterization of Rifampicin Samples and Its Biopharmaceutic Relevance. Eur. J. Pharm. Sci. 2004, 22 (2), 127-144.

Scanning electron microscopy (SEM) was performed using an FEI Quanta 650 instrument (ThermoFisher Scientific, Waltham, MA, USA). Samples were sputter coated with gold coating prior to sample analysis.

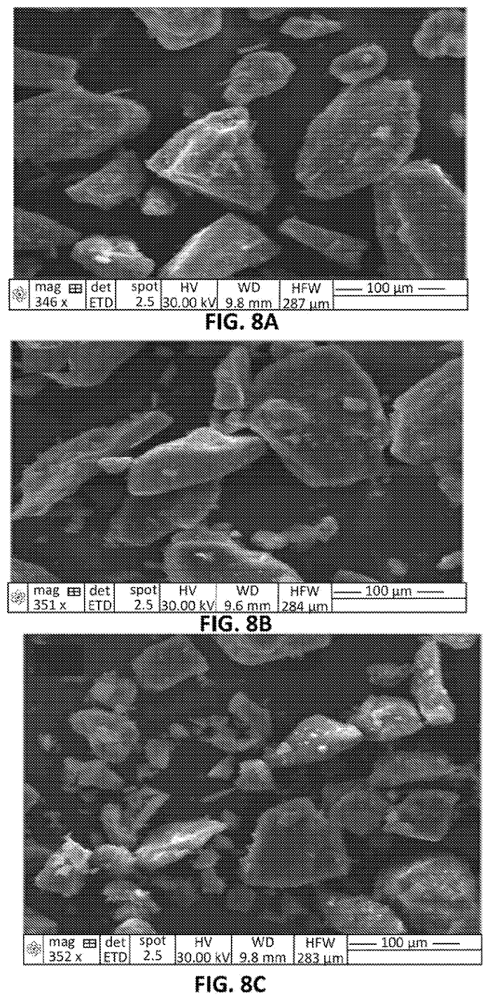

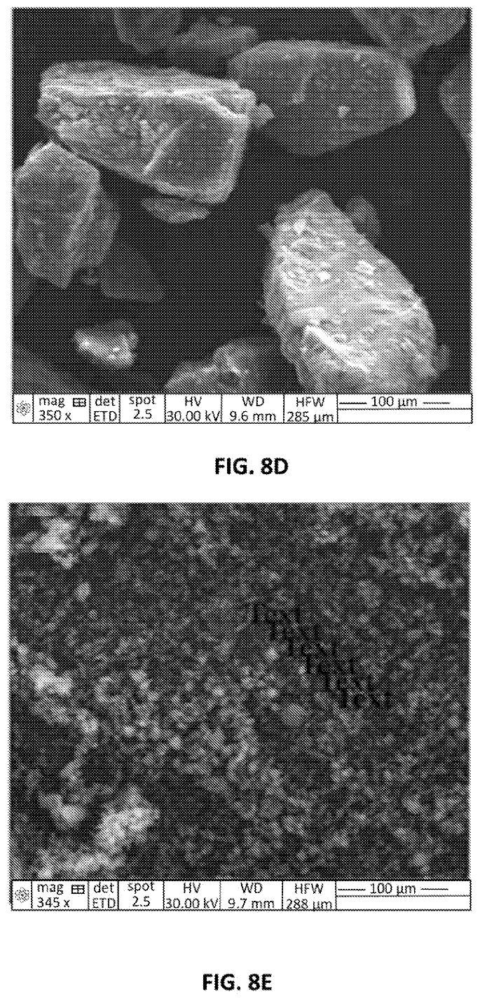

Particle size analysis was conducted on blended materials and compared to pre-blend and lactose/magnesium stearate controls. As shown in Table 3, median sizes (X₅₀) of the extruder blended formulations tended to decrease slightly compared to the pre-blends or the Turbula® blended powders. Similarly, the large particle fraction (X₉₀) of the extruder blended formulation with 4 cycles was lower than other blends. Electron microscopy conducted on the powders also indicated that some morphological changes may result from twin-screw processing (FIGS. 8A-8E). Specifically larger numbers of smaller lactose particles were visible and, with two and four passes through the extruder more rounded edges of lactose were visible compared to the pre-blend or Turbula® mixed powders.

TABLE 3
Particle size distribution for starting materials, twin-screw, and Turbula ® blends.

| | | | | | | Span |
|---|---|---|---|---|---|---|
| Excipient/Formulation | X₁₀ | X₅₀ | | X₉₀ | | ((X₉₀-X₁₀)/X₅₀) |
| Lactohale 206 | 1.91 μm | 4.15 | μm | 116.14 | μm | 27.53 μm |
| Magnesium stearate | 2.08 μm | 8.14 | μm | 17.34 | μm | 1.87 μm |
| Lactohale 206, 0.4% | 5.33 μm | 54.76 | μm | 101.41 | μm | 1.75 μm |
| magnesium stearate | | | | | | |
| Micronized rifampicin | 0.72 μm | 2.18 | μm | 6.42 | μm | 2.61 μm |
| 1% rifampicin, 0.4% | 7.53 μm | 50.47 | μm | 85.22 | μm | 1.54 μm |
| magnesium stearate in | | | | | | |
| Lactohale 206, pre- blend | | | | | | |
| 1% rifampicin, 0.4% | 4.10 μm | 47.67 | μm | 86.71 | μm | 1.73 μm |
| magnesium stearate in | | | | | | |
| Lactohale 206, extruder | | | | | | |
| blended x2 | | | | | | |
| 1% rifampicin, 0.4% | 2.34 μm | 38.26 | μm | 81.83 | μm | 2.08 μm |
| magnesium stearate in | | | | | | |
| Lactohale 206, extruder | | | | | | |
| blended x4 | | | | | | |
| 1% rifampicin, 0.4% | 7.21 μm | 54.00 | μm | 93.34 | μm | 1.60 μm |
| magnesium stearate in | | | | | | |
| Lactohale 206, Turbula ® | | | | | | |
| blended | | | | | | |

### Discussion

Pharmaceutical batch manufacturing is a multi-step, and often lengthy process that has inefficiencies and frequent difficulties in ensuring low variation between batches. Regulatory agencies are encouraging the pharmaceutical industry to consider moving away from batch manufacturing to more controllable and efficient continuous manufacturing. The results presented herein demonstrate the feasibility of utilizing twin-screw extruder technology for powder mixing, which is a critical manufacturing step of dry powder inhalers. Importantly, the results demonstrate that twin-screw mixing is able to blend dry powders for inhalation without decreasing aerosol performance or leading to demixing. From the results presented in this example, utilizing a common carrier system of lactose and magnesium stearate, rifampicin at low concentrations was successfully blended with excellent blend uniformity using this technique. The addition of magnesium stearate for this powder system helped to achieve continuous powder conveyance through the barrel. Thus, the build-up of powder was avoided using this lubricant as the mixture was processed in the low flight clearance co-rotating screw and barrel geometries of the extruder.

Twin-screw mixing achieved better blend uniformity than any time point sampled for Turbula® mixing. In terms of blend uniformity of the rifampicin:lactose:magnesium stearate system, the low shear blending in the Turbula® mixer revealed that this formulation, like other powder formulations, has the tendency to de-mix. In particular, when components in a formulation exhibit strong cohesive forces particle segregation and heterogeneity during mixing may occur. In the case of rifampicin, it appears to have relatively low surface energy and is not strongly adhesive with lactose/magnesium stearate. Accordingly, differences in mixing due to processing differences will be detectable and not obscured by rapid adhesion and mixing of the micronized component to the lactose carrier.

During twin-screw mixing, a relatively small amount of the formulation is processed at any given time in a first-in first-out manner. This is an important potential advantage in critical blending processes. All powder, as it moves through the defined geometry and mechanical movement within the twin-screw extruder, will be subject to same forces for the same time period. This uniformity of forces exerted upon the powder as a whole enables better control over the mixture performance.

Comparing the aerosol performance of twin-screw and Turbula® mixed blends shows that twin-screw achieves superior fine particle fractions. Surprisingly, aerosol performance increased with increasing processing time in the extruder. Batch blending processes have been previously reported to decrease aerosol performance with increasing processing times. From previous studies in Turbula® mixer prepared blends, it has been shown that as mixing energy increases, external press-on forces and drug adhesion to surfaces also increases. Previous studies on mixing budesonide on to lactose carrier particles have shown that as mixing time increases, there is a tendency for drug aggregation on the lactose surface. It has also been indicated in the industry that by increasing mixing time or energy, drug particles bound to the cavities on lactose carrier surfaces are redistributed over the carrier surface where they come in reach of press-on forces.

With twin-screw blending in the studies presented herein, however, the opposite trend is shown. With increasing blending time, increases in dispersion and aerosol performance are observed. Since the powder blends are conveyed and mixed in relatively small masses distributed along the length of the barrel of the twin-screw extruder, powder avalanching and press-on forces are minimized compared to that observed in closed containers comprising the entire batch of powder. In the Turbula® mixer, despite being classified as low shear, the tumbling and avalanching motion of powder from end to end of the container will result in press-on forces greater than the total weight of the powder batch across the cross-sectional area of the container. In addition, some degree of particle size reduction of the formulation was observed as it was processed in the extruder. The larger lactose particles may fracture as they pass through the narrow clearance between the screw and barrel. Many prior reports have detailed how small carrier particle sizes can promote aerosol performance. However, the slight reduction in particle size observed in these studies is unlikely to fully explain the increases in aerosol performance observed. Additionally, particle size reduction during mixing would imply that strong shear forces may existing within the extruder which would be expected to result in strong press-on forces between particles in the mixture. Thus, size reduction observed with laser diffraction analysis of the dispersed powders (e.g., using the Rodos™ system) primarily represents improved particle deaggregation rather than significant milling of the particles. Further, the increased processing times also result in more uniform distribution of cohesive rifampicin across the lactose (less aggregates of rifampicin) and increased pacification of high energy sites on the carrier due to improved distribution of magnesium stearate.

Notably, a lowered device retention of the formulation with increasing extruder processing was observed. Essentially, a 50% reduction in device retention was observed from the pre-blend to the final twin-screw blend. Lowered lactose particle sizes observed (D₉₀ was reduced from approximately 85 microns to 83 microns) is unlikely to be causative as the capsule piercings are much larger scale (mm). Improved emptying is attributed to less aggregated powder due to improved drug and magnesium stearate distribution and the resultant improved fluidization expected as this occurs.

### Example 2: Budesonide Blend Preparation

### Budesonide Blend Example Overview

An ordered mixture of 1% budesonide, 0.3% magnesium stearate, and 98.7% α-lactose monohydrate was prepared using geometric dilution for tumble mixing and sandwich method for high-shear mixing. For twin-screw mixing, the ingredients were pre-mixed for 10 minutes in a V-shell blender and then split-fed with lactose using two twin-screw volumetric feeders to obtain the same final formulation. The mixture was processed at various combinations of feed rate and screw speed. Samples were taken at predesignated locations and time points and evaluated for uniformity. Residence time distribution of the powder in the twin-screw mixer was determined by feeding a small amount of tracer and assessing distribution at the exit end using post-hoc video analysis in MATLAB.

Mixtures were aerosolized using a high-resistance RS01 inhaler (Plastiape) at 60 L/min flow rate and 4 kPa pressure drop across the device. A 30 mg portion of each mixture was filled into a size 3 hydroxypropyl methylcellulose (HPMC) capsule and loaded into the inhaler. The device was connected to a fast-screening impactor (FSI) for size separation followed by vacuum pump.

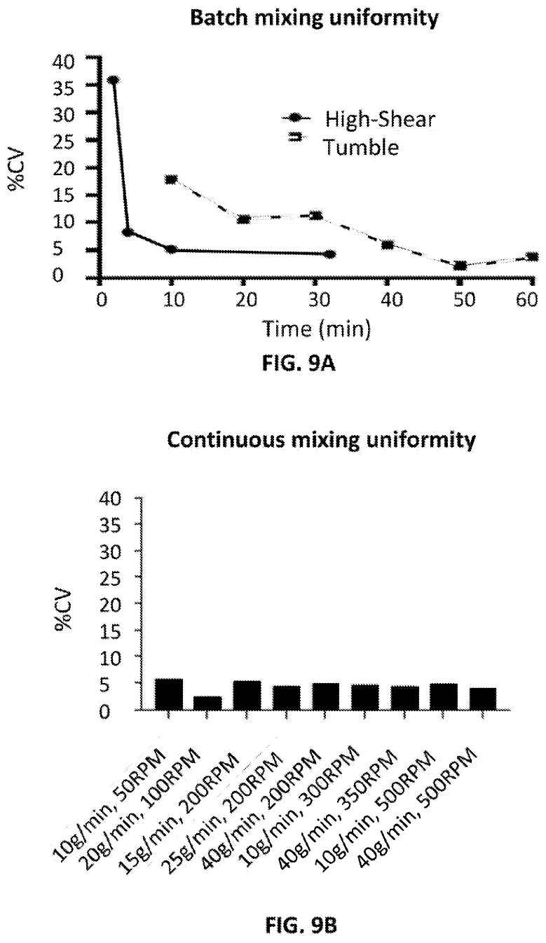

FIGS. 9A-9B summarizes the mixing uniformity of both batch (FIG. 9A) and the twin-screw (FIG. 9B) mixing processes. Spatial distribution of budesonide is highly dependent on mixing time for both tumble and high-shear batch mixing methods. The rapid decrease in drug content variation in the high-shear mixing plot contrasted with the more gradual decrease observed for low shear mixing. This indicates greater mixing energy input due to higher shear generated during mixing. Thalberg, K., et al., Controlling the performance of adhesive mixtures for inhalation using mixing energy. Int J Pharm, 2021. 592: p. 120055. Coefficient of variation (CV) values of less than 5% were obtained following 50 minutes of tumble mixing or 10 minutes of high-shear mixing (FIG. 9A). For twin-screw mixing, the % CV values were less than 5% for all combinations of screw speed and feed rate (FIG. 9B).

Residence time distribution parameters were obtained from the MATLAB modelling software and are summarized in Table 4. Time at which trace first exits the die (Tₛₜₐᵣₜ), the time at which half of the tracer has exited the mixer (T₅₀), and the time at which 90% of the tracer has exited the mixer (T₉₀) were determined from the Fokker-Planck equation that describes the exit-age distribution of the twin-screw mixer. T₉₀ values ranged from 15 seconds to 81 seconds, indicating short residence times for all combinations of processing parameters tested.

TABLE 4
Residence time distribution (RTD) parameters
extrapolated from modeling software.

| Processing parameters | Tₛₜₐᵣₜ(s) | T₅₀ (s) | T₉₀ (s) |
|---|---|---|---|
| 10 g/min, 50 rpm | 50.5 | 71.9 | 81.3 |
| 25 g/min, 200 rpm | 7.5 | 12.9 | 15.6 |
| 10 g/min, 500 rpm | 1.6 | 8.4 | 15.4 |

The intermeshing and over-flight regions of the co-rotating screws allow for efficient dispersive and distributive mixing to reduce agglomerates and form a homogeneous product within a short time frame. Martin, C., Twin Screw Extruders as Continuous Mixers for Thermal Processing: a Technical and Historical Perspective. AAPS PharmSciTech, 2016. 17 (1): p. 3-19. The large surface area-to-volume ratio results in high energy input into a small amount of material in the process section of the barrel. Dombe, G., et al., Application of Twin Screw Extrusion for Continuous Processing of Energetic Materials. Central European Journal of Energetic Materials. Central European Journal of Energetic Materials, 2015. 12 (3): p. 507-522.

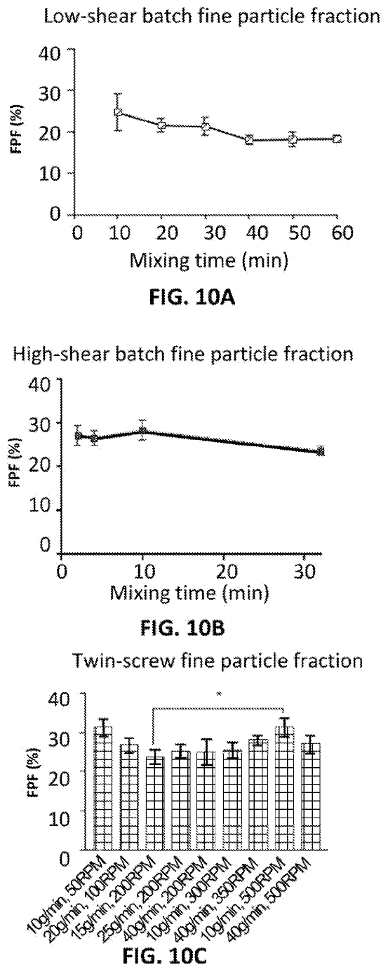

FIGS. 10A-10C show the fine particle fraction (FPF) determined from in-vitro aerosolization experiments at each mixing time for tumble and high-shear batch, and multi-screw continuous mixing. The interactive mixture processed at 15 g/min, 200 rpm produced the lowest FPF of the multi-screw mixtures, which was significantly higher than that of the final blend produced using tumble mixing. Multi-screw FPF values were comparable to those of high-shear batch mixing. No significant differences in FPF were detected amongst all process conditions tested for multi-screw mixing, indicating a robust method for manufacturing DPI mixtures with consistent aerosol performance. Scanning electron microscope (SEM) analysis of all interactive mixtures showed similar morphology across all types of mixing. Particles included tomahawk-shaped carrier with fines dispersed on the surface, which is characteristic of DPI mixtures prepared with sieved lactose. Jones, M. D., et al., The relationship between drug concentration, mixing time, blending order and ternary dry powder inhalation performance. Int J Pharm, 2010. 391 (1-2): p. 137-47.

### Materials and Methods

Budesonide was purchased from Nexconn Pharmatechs (Shenzhen, China). Alpha-lactose monohydrate (InhaLac 230) was obtained from Meggle (Wasserburg, Germany). Magnesium stearate was purchased from Macron Chemicals (Radnor, PA). Microcrystalline cellulose (MCC) PH-200 was secured from Ceolus (Tokyo, Japan) and rhodamine B from Acros Organics (Fair Lawn, NJ). Size 3 hydroxypropyl methylcellulose capsules were a generous gift by Capsugel (Morristown, NJ). 200 proof ethanol was obtained from Decon Labs (King of Prussia, PA), and acetonitrile and methanol from Fisher Chemical (Hampton, NH). All solvents were of analytical grade.

### Micronization of Budesonide

Budesonide was micronized using an Alpine Spiral Jet Mill 50AS (Hosokawa, Summit, NJ). Injector and grinding gas pressures were set to 4.0 bar and 3.7 bar, respectively. Particle size of milled material was confirmed using laser diffraction.

### Particle Size Analysis of Raw Materials

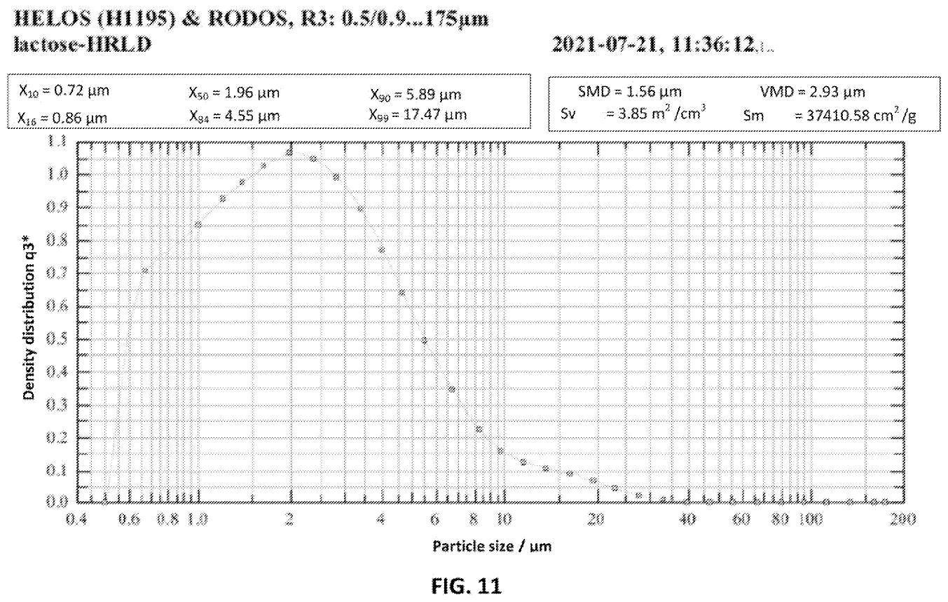

Particle size distribution of alpha-lactose monohydrate, magnesium stearate and budesonide were analyzed using a laser diffractometer (Sympatec, Clausthal-Zellerfeld, Germany). The HELOS detector was equipped with a RODOS dry dispersing unit and R3 (0.9 μm-175 μm) lens. About 0.5 g of each component was carefully loaded on the turntable of the vibratory feeding module. Samples were dispersed at 3 bar and 50% rotation (FIG. 11). Data was acquired using Windox software (Sympatec, Clausthal-Zellerfeld, Germany) and volume-based size distribution was calculated using Fraunhofer theory.

### Composition of Inhalation Mixtures

The same composition of 1% budesonide, 0.3% magnesium stearate and 98.7% lactose was prepared using all three mixing methods.

### Low-Shear Batch Mixing

Prior to mixing, micronized budesonide, magnesium stearate and lactose were separately passed through a sieve with 1 mm opening to remove large agglomerates. Batch size for low shear mixing was determined by matching the amount of powder produced by a 30-minute continuous mixing run at the lowest feed rate, 10 g/min. The volume of low-shear batch mixture prepared was chosen to obtain 50% fill in the mixing vessel. For low-shear mixing, 12 g budesonide and 3.6 g magnesium stearate were sandwiched between 104.4 g of lactose in an 8 oz. stainless steel container to prepare a concentrated mixture. The formulation was mixed for 5 min at 46 rpm using a Turbula orbital mixer (Glen Mills, Clifton, NJ). To prepare the final product, 30 g of the concentrated mixture was sandwiched between 270 g of lactose and mixed at 46 rpm. 30 mg samples were pulled from designated locations (n=3) via micro-sample thief in 10-minute intervals for further analysis of drug content and mixture homogeneity.

### High-Shear Batch Mixing

For high-shear mixing, the batch size was set to 1.4 kg to maintain 50% fill in the mixing bowl. 1381.8 g sieved lactose and 4.2 g magnesium stearate were combined in the bowl of a 4 qt vertical cutter mixer equipped with smooth edge blade (Robot Coupe, Ridgeland, MS). The two components were briefly mixed for 1 min at 500 rpm. Approximately half of the mixture was removed, and 14 g of budesonide was added. The remaining mixture was returned, and the total content was mixed again at 500 rpm. Samples were removed (n=8) at 2, 4, 10 and 32 min for drug content and uniformity analysis. Hertel, M., et al., The influence of high shear mixing on ternary dry powder inhaler formulations. Int J Pharm, 2017. 534 (1-2): p. 242-250.

### Continuous Mixing

The third mixing technique assessed was using a Nano-16 co-rotating twin-screw extruder as a continuous mixer (Leistritz, Somerville, NJ). Batch sizes are not applicable for continuous mixing as mixer volume is no longer a limiting factor. Materials were split fed using two twin-screw volumetric feeders (Brabender, Mississauga, Canada). The surfaces of the drug feeder were coated with a thin layer of polytetrafluoroethylene (PTFE) to minimize budesonide adhesion to the feeder screws.

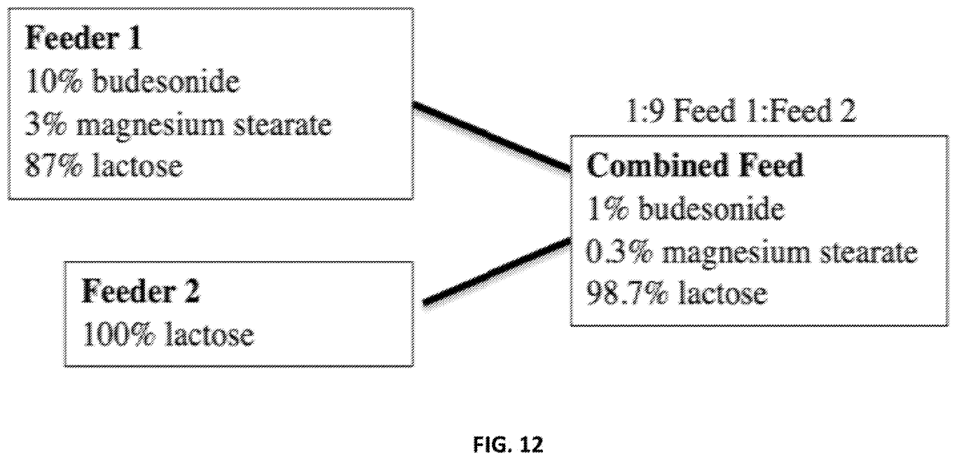

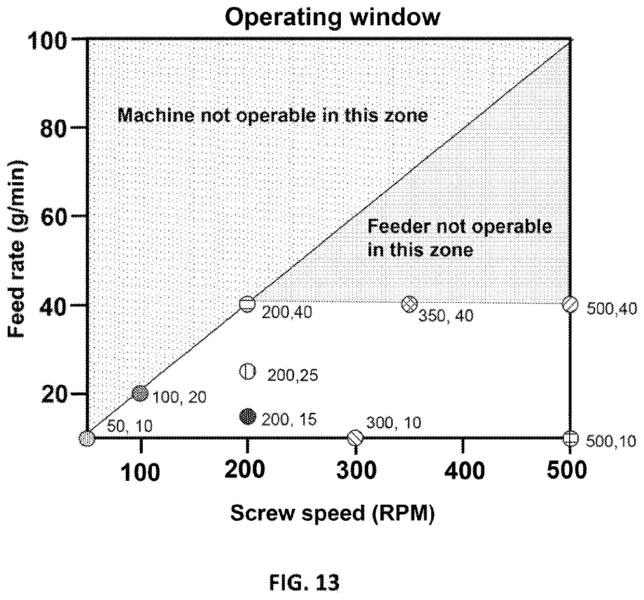

Two mixtures were split fed using two volumetric twin-screw feeders. A diagram of the feeding arrangement is illustrated in FIG. 12. A pre-mixture (termed “feeder 1”) of 10% micronized budesonide, 3% magnesium stearate, and 87% lactose was first prepared by mixing ingredients for 10 min at 25 rpm in a 2 qt V-shell (GlobePharma, New Brunswick, NJ). Feeder 1 was split-fed with lactose in feeder 2 at a 1:9 ratio to obtain a final budesonide concentration of 1%. The extruder was fitted with a screw profile including forward conveying elements and one set of GFM combing mixing elements. The temperature of all three process zones was set to 25° C. while screw speed and feed rate were varied, and powder was collected for further evaluation. FIG. 13 outlines the allowed operating window based on extruder and twin-screw feeder limits. The width of the window is defined by maximum and minimum screw speed limits of 500 rpm and 50 rpm, respectively. The blue shaded region excludes feed rates greater than 40 g/min based on the maximum speed setting on the twin-screw feeder. The red region denotes the non-operable zone of the extruder, which is bound by the diagonal line representing the maximum ratio of feed rate to screw speed, or specific throughput. Specific throughput values greater than 0.2 g/min/rpm will cause torque overload in the Nano-16 for this specific formulation. Operating window limits are a function of screw profile, machine size and material properties. Processing conditions as well as measured torque are given in Table 5. Upon discharging from the extruder, the powder was collected on a conveyor belt (Dorner, Hartland, WI) to allow for in-line sampling. For each run, the first 5 minutes of powder was discarded to allow the process to reach steady state before being collected for 30 minutes. 30 mg samples equivalent to one dose were taken at 10-minute intervals along the conveyor.

TABLE 5
Instrument and processing parameters for continuous mixing and measured torque

| | Zone 1 | Zone 2 | Zone 3 | Feed | Screw | |
|---|---|---|---|---|---|---|
| | Temperature | Temperature | Temperature | rate | speed | Torque |
| Formulation | (° C.) | (° C.) | (° C.) | (g/min) | (RPM) | (G.m.) |
| 1% | 25 | 25 | 25 | 10 | 50 | 40-50 |
| budesonide | 25 | 25 | 25 | 20 | 100 | 60-70 |
| 0.3% | 25 | 25 | 25 | 15 | 200 | 50-60 |
| magnesium | 25 | 25 | 25 | 25 | 200 | 70-75 |
| stearate | 25 | 25 | 25 | 40 | 200 | 60-70 |
| 98.7% lactose | 25 | 25 | 25 | 10 | 300 | 100-110 |
| | 25 | 25 | 25 | 40 | 350 | 80-90 |
| | 25 | 25 | 25 | 40 | 500 | 95-100 |
| | 25 | 25 | 25 | 10 | 500 | 110-120 |

Budesonide was quantified using high-performance liquid chromatography (REF method), and relative standard deviation (RSD) used as a measure of mixture homogeneity. Mixtures with 6% or less variability were taken as uniform. Jones, M. D., et al., The relationship between drug concentration, mixing time, blending order and ternary dry powder inhalation performance. Int J Pharm, 2010. 391 (1-2): p. 137-47.

### Residence Time Distribution (RTD) Analysis

To determine mean residence time of feed material in the twin-screw mixer along with several other measures, a concentrated dye mixture was introduced as a pulse injection and tracer distribution was monitored using a video camera positioned at the discharge end. Tracer was prepared by combining 0.75% w/w rhodamine B with MCC in 50 mL methanol to dissolve the dye. The mixture was left to dry overnight in a ventilated hood and the dried material crushed with a mortar & pestle to remove agglomerates. Three combinations of screw speed and feed rate were selected to estimate a low, middle, and high value for mean residence time. Tracer was added to the feed stream at dye equivalent of 1% of mass flow rate per second to ensure adequate signal-to-noise ratio without oversaturating the color detection channels. Post-hoc video analysis was conducted using MATLAB (The Math Works, Natick, MA) by fitting the color readings with an analytical solution. Briefly, a region of interest (ROI) was selected for analysis from the recorded video. The color intensity of the tracer in the ROI was analyzed for each frame in the video to monitor the change in color over time. The RTD curve was fitted to the solution of the Fokker-Planck equation:

$$E ( τ ) = \sqrt{\frac{P e}{π τ}} exp ( - \frac{Pe}{4} \frac{{( 1 - τ )}^{2}}{τ} ) - \frac{P e}{2} exp ( Pe ) erfc ( \sqrt{\frac{Pe}{4}} \frac{1 + τ}{\sqrt{τ}} )$$

where Pe=Peclet number and

$$τ = \frac{τ}{θ} ,$$

where t=time and θ=mean residence time. Wahl, P. R., et al., In-line measurement of residence time distribution in melt extrusion via video analysis. Polymer Engineering & Science, 2018. 58 (2): p. 170-179. Several measures were derived from the RTD fit curve and compared across mixing conditions to assess the quality of mixing and compare the mixing times of both batch processes.

### Determination of In Vitro Aerosol Performance

Aerodynamic particle size was evaluated with the high resistance RS01 inhaler (Plastiape S.p.a, Osnago, Italy) and fast-screening impactor (Copley Scientific, Nottingham, UK). The abbreviated impactor was fitted with a 60 L/min insert and glass fiber filter to collect the coarse and fine fractions, respectively. A flow rate of 60 L/min was set to achieve an approximate pressure drop of 4 kPa across the device and air was drawn for 4 s to ensure 4 L inhalation volume. One capsule was actuated per run, and each mixture was tested in triplicate. Following actuation, each component of the apparatus was rinsed with known volumes of solvent. Fine particle dose (FPD) was taken as the drug mass <5 μm deposited on the filter. Fine particle fraction (FPF) was calculated as the ratio of fine particle dose to total recovered dose.

### HPLC Quantification of Budesonide

Budesonide was assayed using an HPLC (Waters Corp, Milford, MA) equipped with a Luna C18 150×4.6 mm column (Phenomenex, Torrance, CA). All samples were prepared in 80 proof ethanol while the mobile phase was 40:60 H2O:acetonitrile (v/v) at a flow rate of 1 mL/min. Budesonide was analyzed at a wavelength of 244 nm with a photodiode array (PDA) detector (Waters Corp, Milford, MA).

### Scanning Electron Microscopy (SEM)

The size and morphology of both raw materials and produced mixtures were evaluated using high-vacuum scanning electron microscopy (FEI, Hillsboro, OR). Samples were mounted on aluminum stubs with double sided carbon tape and sputter coated with gold for 1 minute (Electron Microscopy Sciences, Hatfield, PA). Sample-specific magnifications and voltages ranging from 10 kV-30 kV were used to view the specimens.

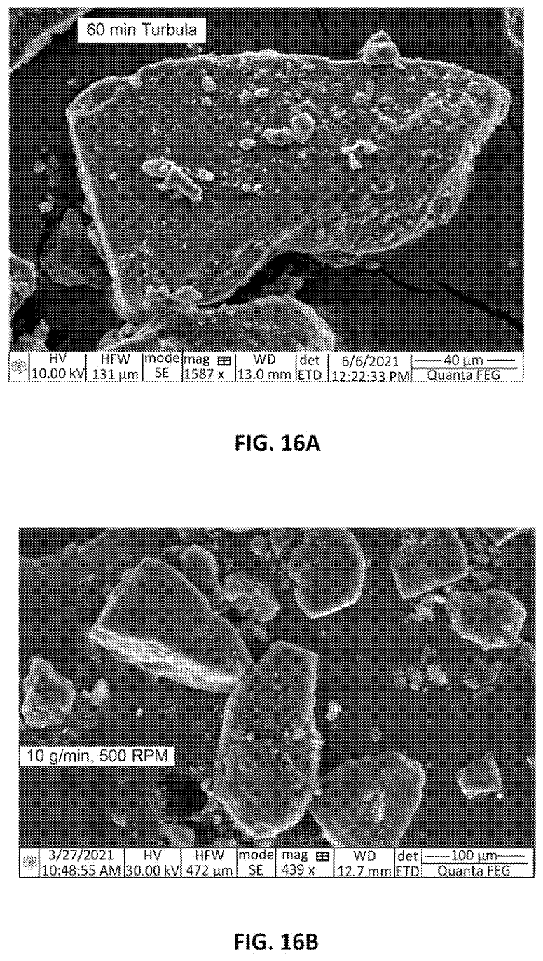

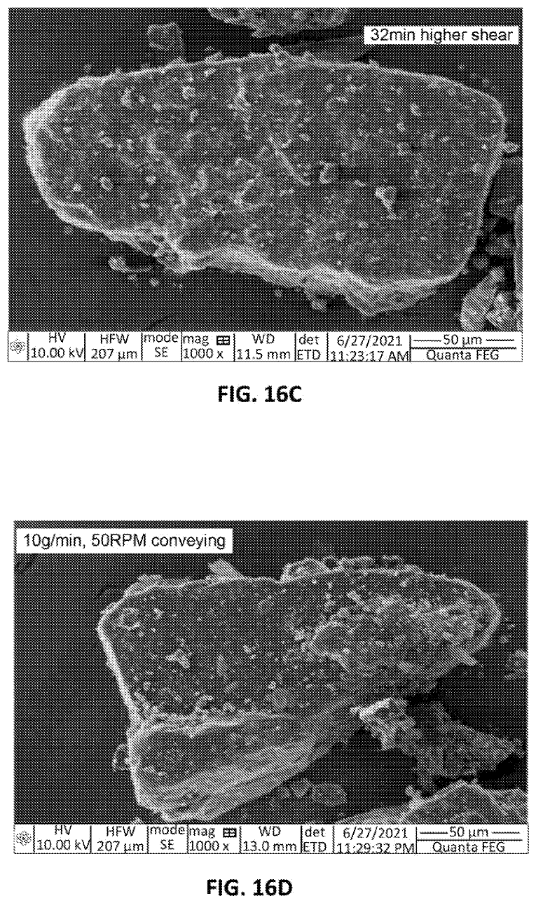

Scanning electron micrographs of the adhesive mixtures revealed large, wedge-like particles >100 μm in length like the original tomahawk shape of commercial sieved lactose (FIGS. 16A-16D). Smaller particles and agglomerates ranging from 5-15 μm appear to be dispersed on the surface of the carrier on both smooth surfaces and indentations, confirming that there was some level of adhesive interaction present in the three mixing techniques. Samples taken from the low-shear batch mixture at the 60-minute time point show carrier particles with loosely attached agglomerates likely including a mixture of budesonide, magnesium stearate and lactose. As mixing time increases, the material protruding from the flat carrier surfaces flattens and covers a larger region. For high-shear batch samples, the material deposited on lactose is considerably smaller in size and achieves a more visually uniform dispersion. A smearing effect is visible in the 32-minute sample with a few agglomerates. The morphology of most continuous mixtures shows similar tomahawk shaped carriers apart from the 10 g/min, 500 rpm and 10 g/min, 300 rpm mixtures. Irregularly shaped particles with sharp edges and sizes ranging from 25-100 μm predominate, which suggests the occurrence of particle attrition during the mixing process. For all mixtures, the fines preferentially adhered to the rough, indented regions of lactose with lower concentrations on smooth regions. SEM confirms that twin-screw mixing produces adhesive mixtures with similar morphology to those of low-shear and high-shear batch methods.

### Analysis of Drug Form Change in Twin-Screw Produced Powder

A concentrated slurry of micronized drug and magnesium stearate was produced by adding water to the DPI powder to dissolve lactose. The mixture was centrifuged at 7000 rpm for 5 minutes and the supernatant was removed. This procedure was repeated three more times to rinse away trace lactose. The wet pellet was mounted on a glass XRD slide and diffraction patterns were obtained using Miniflex II XRD (Rigaku, Tokyo, Japan) and compared to pure drug. The XRD was operated at a voltage of 40 kV, 15 mA current. Samples were scanned using 2-theta angle range of 3-50 with a 0.02 step size and 0.4 s dwell time.

### Statistical Analysis

A one-way analysis of variance (ANOVA) was performed using JMP statistical software (SAS, Cary, NC) to detect significant differences in aerosol performance. Dunnett's (with control) test was used to determine outliers with an alpha level of 0.05. Least-squares regression was implemented to fit a predictive trend between extruder parameters and dispersion quality.

### Particle Size of Starting Materials

Table 6 lists the particle size distribution for starting materials as determined by laser diffraction. Budesonide was confirmed to be sufficiently micronized (e.g. less than 5 μm). Magnesium stearate and lactose size distributions were within the size ranges provided by manufacturers.

TABLE 6
Particle size distribution (PSD) of starting
material using RODOS laser diffraction.

| Material | D10 (μm) | D50 (μm) | D90 (μm) |
|---|---|---|---|
| Micronized budesonide | 0.69 | 1.68 | 4.39 |
| Lactose | 32.09 | 80.34 | 111.84 |
| Magnesium stearate | 1.29 | 4.45 | 9.93 |

### Mixture Homogeneity and Residence Time Distribution

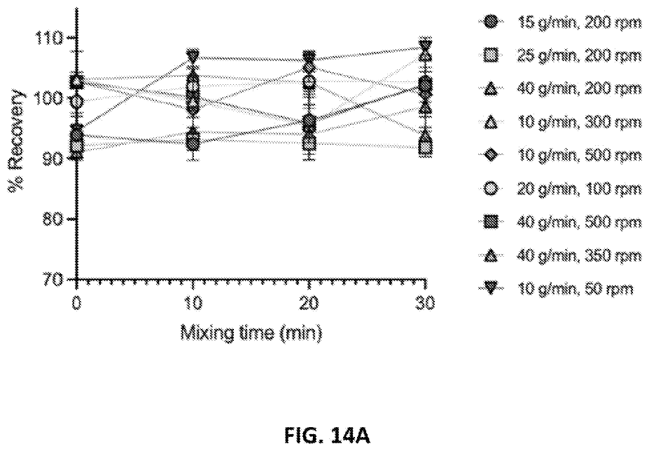

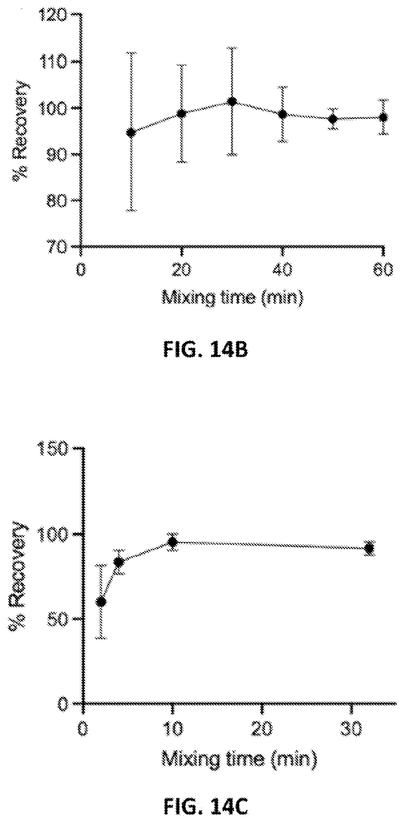

Recovery was calculated as % of actual drug amount relative to nominal amount for each location sampled during mixing. % Recovery as a function of time was plotted for each mixing technique (FIG. 14A). Plots of % RSD versus mixing time were determined for both low and high-shear batch mixing (FIG. 14B and FIG. 14C). % RSD was less than 6% after 40 minutes of tumble mixing, and 10 minutes of high-shear mixing. Positive and negative rates of change in RSD are shown for low-shear batch mixing. For high-shear batch mixing, initial recovery was low at about 60% of theoretical, but gradually increased to over 90% after 10 minutes (FIG. 14C). Drug recovery values at each timepoint during continuous mixing were averaged and % RSD was plotted for each processing condition (feed rate, screw speed). % RSD values were less than 6% for all continuously mixed powders. Residence time distribution parameters were obtained from the MATLAB modelling software and are summarized in Table 4. Time at which trace first exits the die (Tₛₜₐᵣₜ), time at which half of the tracer has exited the mixer (T₅₀), and time at which 90% of the tracer has exited the mixer (T₉₀) were determined from the Fokker-Planck equation that describes the exit-age distribution of the win-screw mixer. T₉₀ values ranged from 15 seconds to 81 seconds. Not intending to be bound by theory, these T₉₀ values suggest short residence times for all combinations of processing parameters tested.

### In Vitro Aerosol Performance

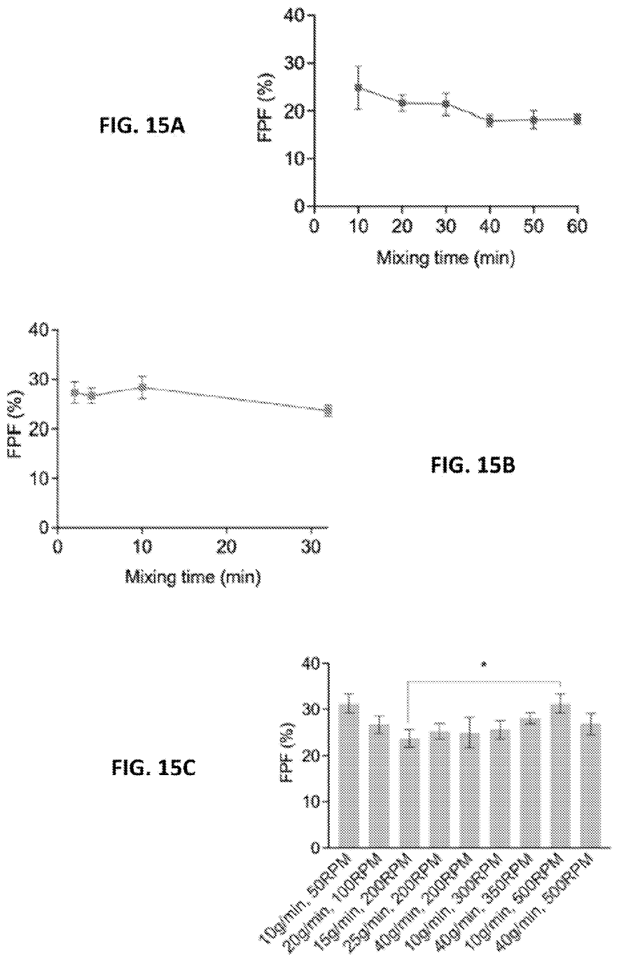

PFP values were plotted as a function of mixing time for both batch mixing processes (FIG. 15A and FIG. 15B). FPF decreases with mixing time for low-shear batch mixing. FPF of the high-shear batch final powder is greater than that of low-shear batch with lower % RSD. FIG. 15C also shows FPF for each twin screw processing condition. Average values range from 23.7%-31.3% for the screw speed and feed rate combinations tested. A significant increase in FPF was detected from the 15 g/min, 200 rpm to 10 g/min, 500 rpm condition. The 15 g/min, 200 rpm powder had the lowest FPF (23.7%), which was higher than that of the final low-shear batch mixture (18.3%) and comparable to that of the final high-shear batch mixture (23.6%). Final blend RSD was 5% for low and high-shear batch and 4-13% for twin screw powders.

### Statistical Evaluation of Continuous Mixing Data

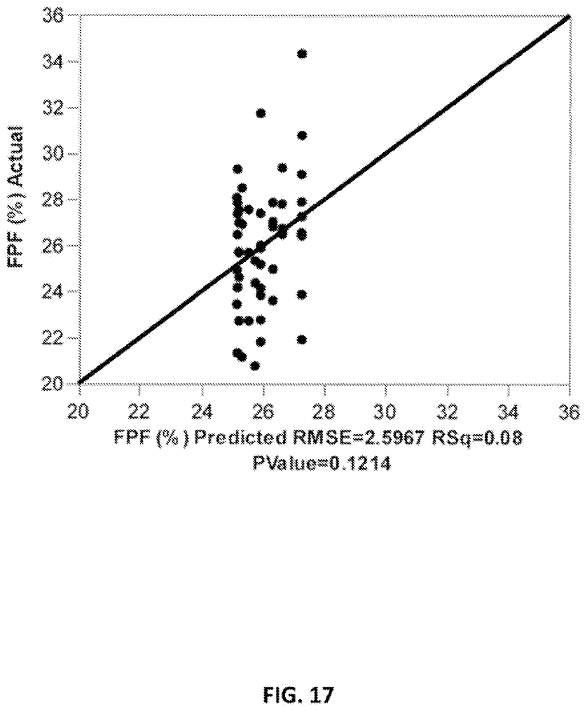

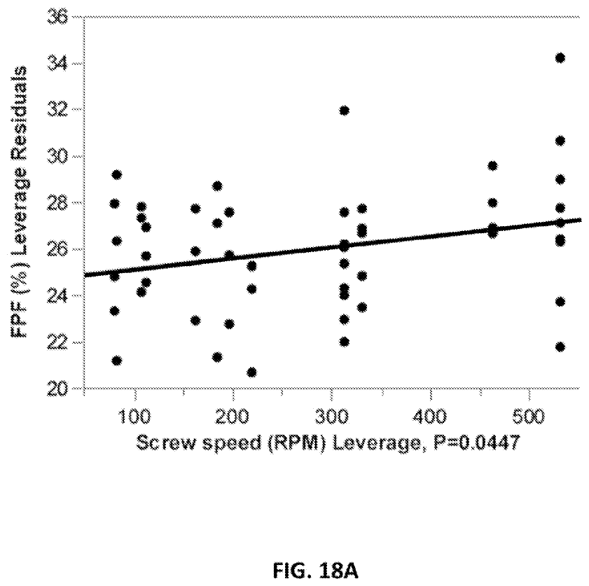

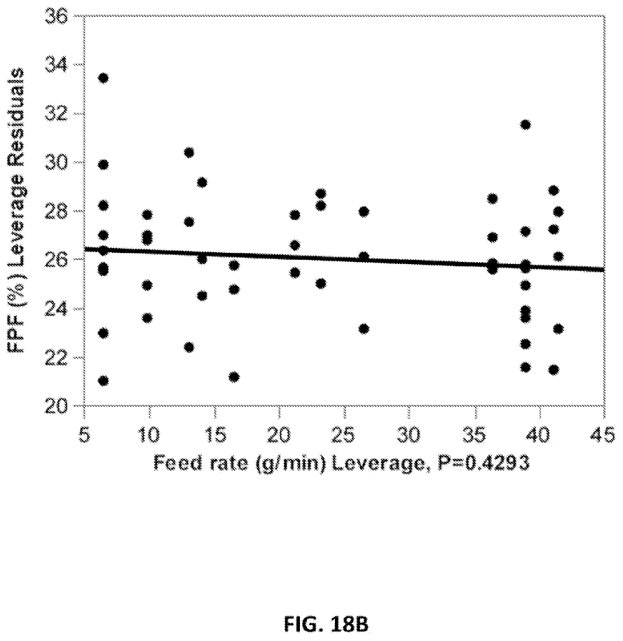

The aerosol dispersion data obtained from continuous mixing was analyzed using least-squares regression to generate a fit model relating processing parameters to product performance (FIG. 17). Variables included were screw speed and feed rate while FPF served as the inhalation performance metric. The R2 value of the regression model was 0.08. This suggests that a relationship between screw speed, feed rate and FPF cannot be detected within the limits of the operating window. Leverage plots were also produced that analyze the relative importance of each factor on the predictive model (FIG. 18). No significant influence of screw speed or feed rate on the aerosol performance was observed.

### Discussion

Analysis of the twin-screw samples taken from the conveyor showed that for all processing conditions, budesonide was fully recovered, and distribution was uniform. The high surface area to volume ratio of the processing zone allows for extremely efficient mixing of materials. Grooved mixing (GFM) elements provides more distributive and dispersive mixing than conveying elements, while they provide less dispersive mixing than kneading elements. The high viscosity of polymers requires kneading elements to generate viscous heat dissipation and dispersive mixing during processing. However, GFM elements are ideal for preparing inhalation powders since excessive press-on force by generated by kneading elements could be detrimental to aerosol performance. For adhesive mixtures, particles in the intermeshing and over flight regions are exposed to high shear to break up drug agglomerates and disperse fines onto lactose.

A comparison of low-shear and high-shear batch mixing revealed the sensitivity of the drug homogeneity to mixing time. A general trend toward lower variability with increasing mixing time was seen for both methods; however, high-shear batch mixing was considerably less variable than low-shear for this particular composition. The low budesonide recovery in early time points indicates that the mixture is not homogeneous. The difference in mixing behavior between low shear and high shear mixing can be attributed to the rotating blade inside of the high-shear batch mixer, which introduces dispersive forces that effectively break up budesonide agglomerates and disperse the drug fines onto carrier surfaces. The tumbling mechanism of the low-shear batch mixer promotes self-agglomeration of budesonide but insufficient shear is generated to overcome the inherent cohesion. Tumble mixers can produce homogeneous powders when individual components possess similar micromeritic properties but encourage segregation of materials of different particle sizes. The positive and negative rates of changes in % RSD for low-shear batch mixing indicate the occurrence of both mixing and segregation.

FPF varies greatly with mixing configuration due to process-dependent shear. Agglomerates transfer energy due to friction and collision during shearing. With sufficient shear agglomerates will be reduced and adhered to the carrier surface. Insufficient shear results in intact agglomerates which may combine to form larger agglomerates. Delivery of fine particle dose to the lungs relies on efficient separation of the drug from the carrier; therefore, an essential balance between sufficient force applied to adhere the drug to lactose and excessive press-on force that permanently incorporates the drug into the carrier surface exists. Lubricants can be used to increase aerosolization by reducing carrier surface energy. Magnesium stearate reduces van der Waals and electrostatic forces between course and fine excipients, which causes higher drug detachment. During interactive mixing, the lubricant coats lactose and drug fine particles to reduce surface energy and significantly improve aerosolization. This is countered by the incorporation of said fines into the larger lactose particles due to press-on forces generated during mixing. Lubricant level is constant across all three mixing methods studied; however, the mixing configuration varies. Since adhesion forces rely heavily on process-dependent pressure applied to the drug and carrier, the aerosol can behave differently.

The high FPF values of all twin-screw powders as compared to low-shear tumble mixtures indicate that the novel process described herein results in better aerosol performance. When comparing FPF of the different mixing types, mixture homogeneity must be considered. For example, the 10-minute mixture for low-shear batch mixing is the best performing but powder variability is 17%. Therefore, one can only consider uniform mixtures to practically fill an inhaler device. FPF values for all powders processed by twin-screw were similar, except for the highest performing powder (10 g/min, 500 rpm). This highlights the robust nature of the twin-screw mixing process in being able to produce homogeneous mixtures using various mixing conditions without impacting aerosol performance significantly. Twin-screw continuous mixing is highly efficient due to the large surface area-to-volume ratio in the extruder process section. The Nano-16 extruder has a free volume of 1 cc/screw diameter and length-to-diameter ratio of 20:1. One fully filled barrel length of powder equates to 20 mL, whereas process volumes were more than 400 mL for both batch mixers used in this study. The large surface area-to-volume ratio allows for intensive mixing, suitable for cohesive materials like budesonide. In addition, the screw profile can be customized to impart the desired amount of shear. In continuous processing, all regions of the material experience the same mixing history, which results in more consistent product quality.

Critical quality attributes of an optimized DPI formulation include homogeneity and dispersion of 1-5 μm drug particles on the carrier surface. Distributive mixing is necessary to achieve uniform spatial distribution of the mixture components while drug agglomerates are reduced to individual particles via dispersive mixing. The modular nature of the twin-screw continuous mixer allows the mixing configuration to be customized for specific aims. Bulk mixing occurs at the intermeshing region between the at least two co-rotating screws, where the channels of powder from one screw are picked up by the second screw. The high shear environment in the intermeshing region between two screws, and the overflight region between screw flights and the barrel aids in dispersion of larger agglomerates into smaller particle sizes. In this study, the screw profile primarily included conveying elements with the intention to minimize press-on forces. Such screw elements are primarily intended for axial displacement and not high-shear mixing. One set of GFM combing elements was included to introduce distributive and dispersive mixing of the powder streams separated by the screw flights to ensure mixture homogeneity. Kneading elements were not included to avoid excess press-on forces and torque overload.

The aerosol performance of powder produced by low-shear batch mixing does not significantly change over time. As mixing time increases, there is more distributive and dispersive mixing occurring, resulting in more consistent FPF. A trade-off exists between mixing for sufficient time so that the powder becomes homogeneous on a unit-dose level and achieve good release of the respirable particles from the carrier. During mixing, drug and fines can combine to form “soft agglomerates”. These structures can be aerosolized if the ratio of drug to fines is low. If the agglomerates contain a high amount of drug, they may remain intact due to cohesive forces, and reduce FPF. Budesonide is a highly cohesive drug that prefers to self-aggregate rather than interact with lactose. Shear forces generated during the mixing process can help to disperse cohesive materials into smaller particles, for example when there are sufficient forces in the process to overcome the cohesive nature of the agglomerates. A dynamic equilibrium occurs between drug detachment and adhesion to carrier particles. The consistent aerosol performance can be due, in part, to the saturation of high-energy active sites on the carrier with fines including budesonide, lactose, and magnesium stearate. Once all the high-energy active sites are occupied by drug particles, lower-energy regions are filled from which budesonide readily separates during inhalation.

For high-shear batch final product, FPF is higher than that of low-shear batch mixing, and RSD is lower. For low-shear batch mixing, both lubricant and drug were mixed with a portion of lactose to form a concentrated mixture. Geometric dilution of the concentrated mixture with lactose was used to prepare the final product. For high-shear batch mixing, magnesium stearate was first layered onto the carrier during the 1-minute pre-mixing step before budesonide was added in a sandwich method. The pre-mixing step of lactose with lubricant can contribute to the improved aerosol performance in comparison to low-shear batch mixing due to the saturation of active sites and the buffer phenomenon. Fines that are coarser than budesonide particles serve as a buffer during mixing and prevent excess press-on forces. The rotating impeller inside the high-shear batch mixing also imparts shear to disperse drug agglomerates into respirable fractions and give improved aerosol performance.

The advantage of twin-screw mixing lies in the short residence time of the powder in the mixer barrel. The twin-screws convey material forward while new feed material is continuously introduced. The residence time of powder inside the barrel was relatively similar for the three continuous process conditions tested. The initial 10% budesonide feedstock was mixed in the V-shell for 10 minutes prior to feeding into the extruder with lactose. The total mixing time for each twin-screw processing condition is taken as a sum of the feedstock mixing time and the residence time. Therefore, total mixing time for all twin-screw mixing conditions is no more than 12 minutes. Despite this short process time, mixture uniformity analysis confirmed homogeneous powders for all twin-screw conditions. Comparison with batch data reveals that twin-screw can produce uniform mixtures much faster than tumble mixing and at a similar rate as high-shear mixing.

FPF variability is the lowest at the final timepoints for both batch processes and lower than all continuously processed mixtures. Since FPF measures the relative mass of inhaled drug less than 5 μm, the amount of budesonide that deposits on the filter of the FSI depends on both mixture homogeneity and drug particle size. In earlier time points for both batch mixing processes, fluctuations in aerosol performance can be attributed to regions of concentrated drug that have not fully separated into individual particles. As the mixtures become more uniform over time, budesonide is dispersed into homogeneous particle sizes and aerosol performance becomes more consistent. The higher % RSD for twin-screw FPF can be due, for example, to the generation of new high-energy lactose surfaces during processing as well as differences in carrier particle size. Lactose surface properties can significantly influence drug adhesion and consequently aerodynamic behavior. As new surfaces are generated, budesonide may interact differently with these energetic regions to cause variable FPF. This variability can be reduced by increasing the number of mixing elements or using a twin-screw mixer with a longer barrel to increase residence time. Additional studies confirmed that passing the 40 g/min, 200 rpm powder through the continuous mixer two more times reduced variability in FPF from 13% to 6%. This is analogous to having longer process section with multiple mixing zones in which lactose will be exposed to a longer mixing time to generate a more uniform particle size distribution.

When attempting to feed micronized drug alone, drug compaction and adherence to the feeder twin-screws was observed, resulting in decreasing weight output over time. Since aerosol performance relies on particle size and morphology, the presence of large drug agglomerates in the mixture is not ideal. For this reason, a gravimetric feeder was not considered as simply increasing feed rpm to achieve target output would likely worsen compaction. The adhesive interactions between micronized budesonide and different fluoropolymer coatings were assessed but no improvements in flow were seen. The feeding step is crucial for producing accurate drug load and must not be overlooked. Since pure micronized budesonide cannot be fed using a twin-screw feeder, the drug was diluted with free-flowing lactose and magnesium stearate to aid in consistent feeding.

Scanning electron micrographs of the adhesive mixtures revealed large, wedge-like particles >100 μm in length like the original tomahawk shape of commercial sieved lactose. Smaller particles and agglomerates ranging from 5-15 μm appear to be dispersed on the surface of the carrier on both smooth surfaces and indentations, confirming that there was some level of adhesive interaction present in the three mixing techniques. Samples taken from the low-shear batch mixture at the 60-minute time point show carrier particles with loosely attached agglomerates likely including a mixture of budesonide, magnesium stearate and lactose. As mixing time increases, the material protruding from the flat carrier surfaces flattens and covers a larger region. For high-shear batch samples, the material deposited on lactose is considerably smaller in size and achieves a more visually uniform dispersion. A smearing effect is visible in the 32-minute sample with a few agglomerates. The morphology of most continuous mixtures shows similar tomahawk shaped carriers apart from the 10 g/min, 500 rpm and 10 g/min, 300 rpm mixtures. Irregularly shaped particles with sharp edges and sizes ranging from 25-100 μm predominate, which shows the occurrence of particle attrition during the mixing process. For all mixtures, the fines preferentially adhered to the rough, indented regions of lactose with lower concentrations on smooth regions. SEM confirms that twin-screw mixing produces adhesive mixtures with similar morphology to those of low-shear and high-shear batch methods.

### Example 3: Budesonide Low Dose Blend Preparation Using Different Mixing Elements

### Budesonide Blend Example Overview

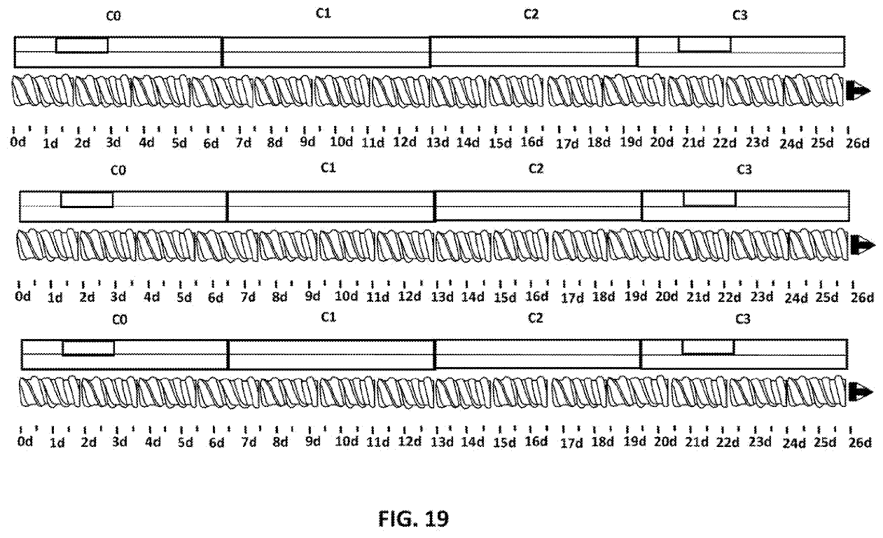

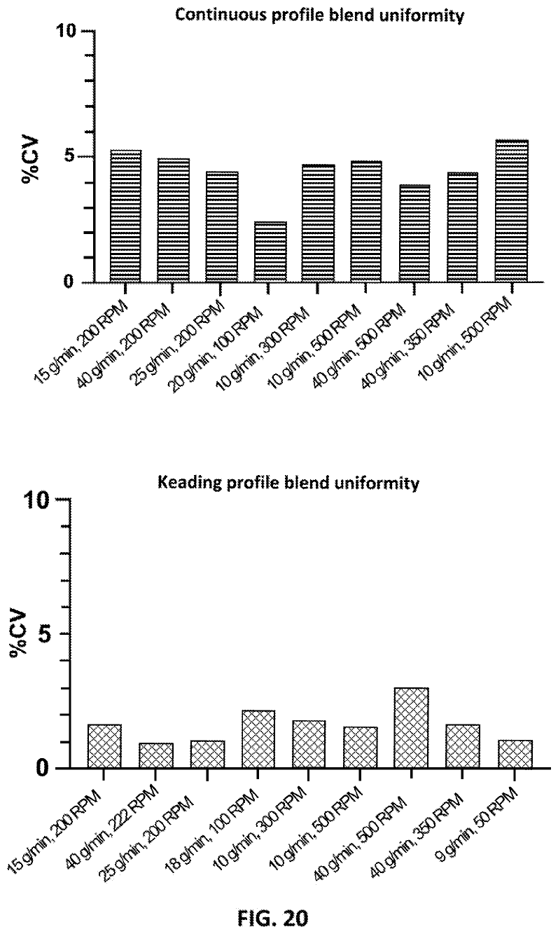

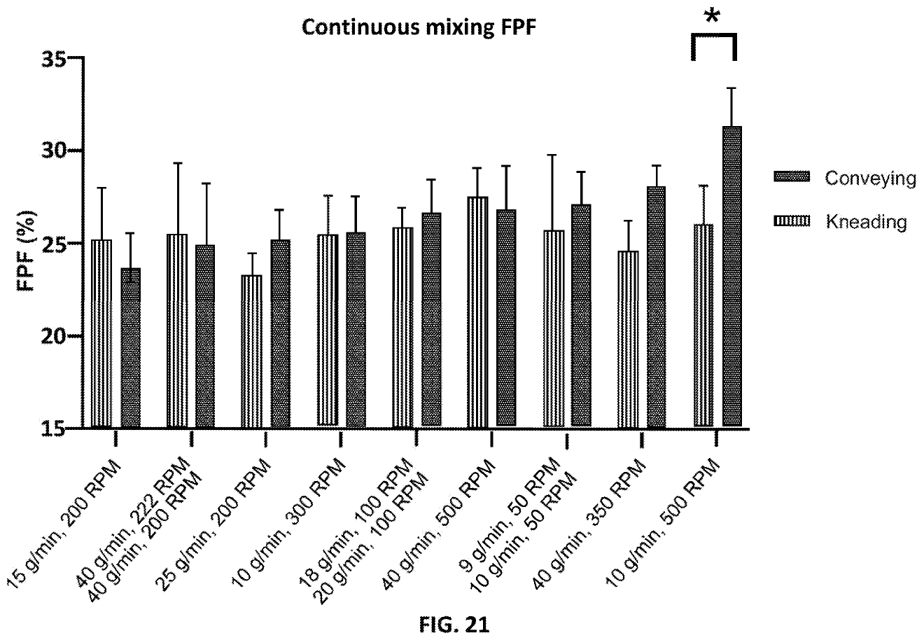

The budesonide blend was prepared using three different mixing elements: (1) a conveying element (FIG. 19, top), (2) a 30-degree element (FIG. 19, middle), and (3) a 60-degree element (FIG. 19, bottom). FIG. 20 summarizes the mixing uniformity of both conveying (left) and the kneading (right) mixing processes. FIG. 21 shows the fine particle fraction (FPF) determined from continuous mixing experiments at different feeding rates and speeds. A significant difference in FPF was observed between the kneading and conveying forms where the mixture was processed at 10 g/min and 500 rpm.

### Materials and Methods

Budesonide was purchased from Nexconn Pharmatechs (Shenzhen, China). Alpha-lactose monohydrate (InhaLac 230) was obtained from Meggle (Wasserburg, Germany). Magnesium stearate was purchased from Macron Chemicals (Radnor, PA). Microcrystalline cellulose (MCC) PH-200 was secured from Ceolus (Tokyo, Japan) and rhodamine B from Acros Organics (Fair Lawn, NJ). Size 3 hydroxypropyl methylcellulose capsules were a generous gift by Capsugel (Morristown, NJ). 200 proof ethanol was obtained from Decon Labs (King of Prussia, PA), and acetonitrile and methanol from Fisher Chemical (Hampton, NH). All solvents were of analytical grade.

### Micronization of Budesonide

Budesonide was micronized using an Alpine Spiral Jet Mill 50AS (Hosokawa, Summit, NJ). Injector and grinding gas pressures were set to 4.0 bar and 3.7 bar, respectively. Particle size of milled material was confirmed using laser diffraction.

### Particle Size Analysis of Raw Materials

Particle size distribution of alpha-lactose monohydrate, magnesium stearate and budesonide were analyzed using a laser diffractometer (Sympatec, Clausthal-Zellerfeld, Germany). The HELOS detector was equipped with a RODOS dry dispersing unit and R3 (0.9 μm-175 μm) lens. About 0.5 g of each component was carefully loaded on the turntable of the vibratory feeding module. Samples were dispersed at 3 bar and 50% rotation. Data was acquired using Windox software (Sympatec, Clausthal-Zellerfeld, Germany) and volume-based size distribution was calculated using Fraunhofer theory.

### Continuous Mixing

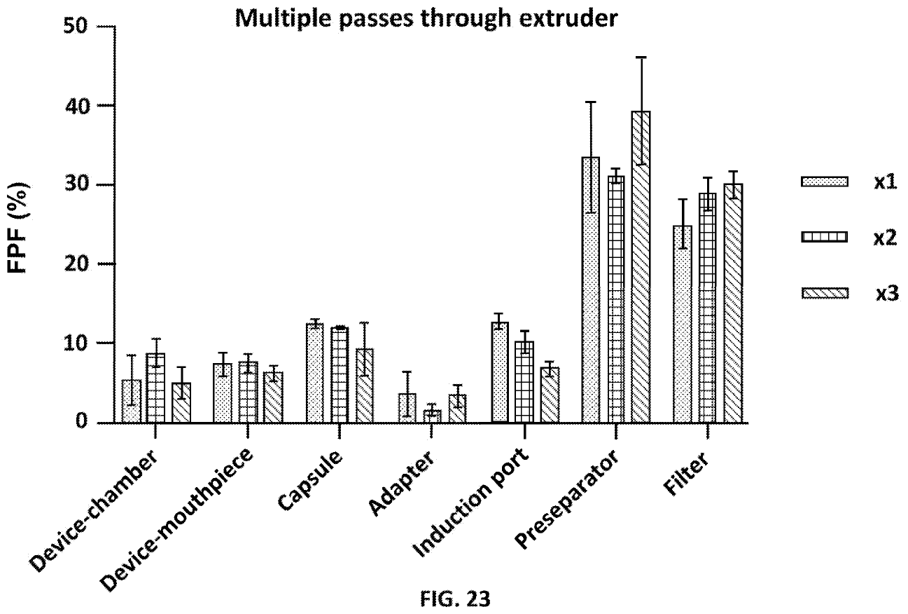

Nano-16 co-rotating twin-screw extruder was used as a continuous mixer (Leistritz, Somerville, NJ). Batch sizes are not applicable for continuous mixing as mixer volume is no longer a limiting factor. Materials were split fed using two twin-screw volumetric feeders (Brabender, Mississauga, Canada). The surfaces of the drug feeder were coated with a thin layer of polytetrafluoroethylene (PTFE) to minimize budesonide adhesion to the feeder screws. The aerosol performance of the powder blend passed through the twin-screw extruder is shown in FIG. 23.

### Scanning Electron Microscopy (SEM)

The size and morphology of both raw materials and produced mixtures were evaluated using high-vacuum scanning electron microscopy (FEI, Hillsboro, OR). Samples were mounted on aluminum stubs with double sided carbon tape and sputter coated with gold for 1 minute (Electron Microscopy Sciences, Hatfield, PA). Sample-specific magnifications and voltages ranging from 10 kV-30 kV were used to view the specimens.

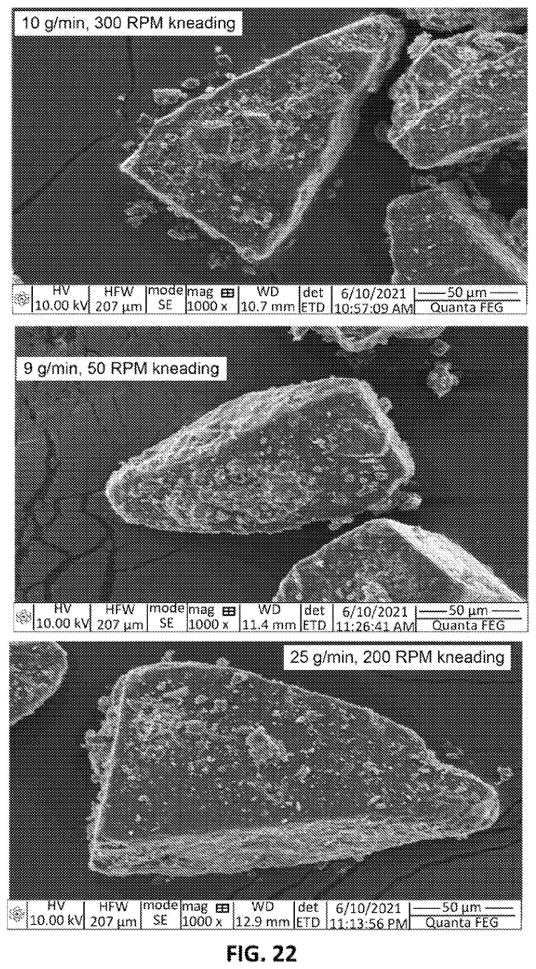

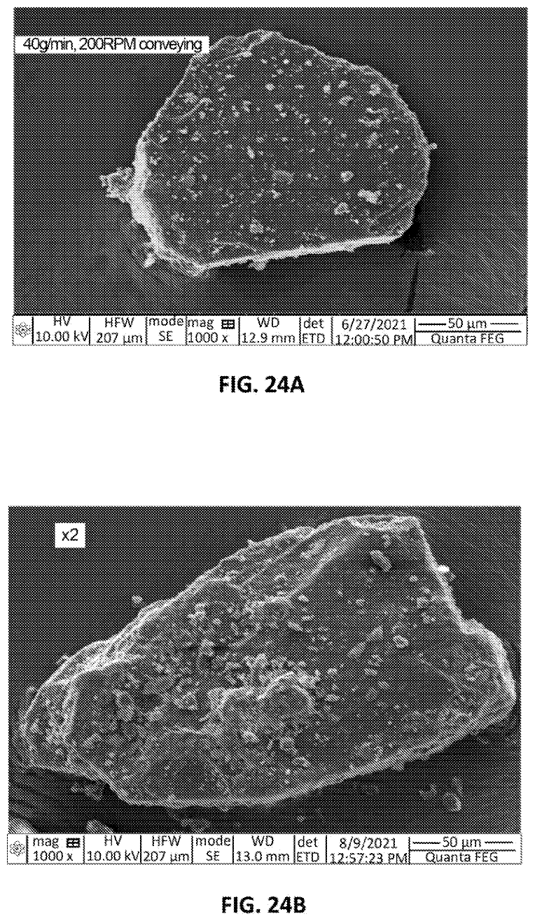

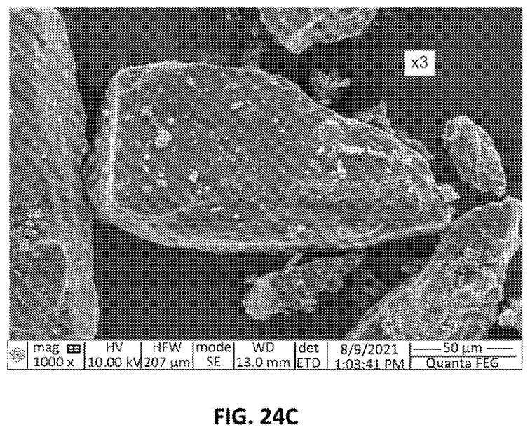

Scanning electron micrographs of the adhesive mixtures revealed large, wedge-like particles >100 μm in length like the original tomahawk shape of commercial sieved lactose for both kneaded (FIG. 22) and conveyed extrusions (FIGS. 24A-24C). Smaller particles and agglomerates ranging from 5-15 μm were dispersed on the surface of the carrier on both smooth surfaces and indentations, confirming that there was some level of adhesive interaction present in kneading and conveying.

### Example 4: Powder Blend for Tablet Manufacturing

A blend of low dose drug is prepared by combining 1% drug, 0.1% silicon dioxide, 40% mannitol, 50% microcrystalline cellulose, 0.9% magnesium stearate, 2% hydroxypropyl cellulose, and 6% croscarmellose sodium. Silicon dioxide is added to improve feeding of the drug into the batch mixer. A batch mixer is used to blend the micronized drug. More specifically, a method of split feeding is used when developing the powder blend for tablet manufacturing in order to better adjust the blending of all elements involved. The order of feeding is as follows: (1) the blend of drug and silicon dioxide is first combined, (2) magnesium stearate is then added, (3) hydroxypropyl cellulose is added next, (4) croscarmellose sodium is added next, (5) mannitol is added next, and (6) microcrystalline cellulose is added last. Each component is fed individually into the feeding hopper of the extruder. The final blend is then collected and used to prepare a tablet including the powder blend.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

### Example 5: Preparing a Budesonide Aerosol Powder Blend

### Materials and Methods

Budesonide was purchased from Nexconn Pharmatechs (Shenzhen, China). Alpha-lactose monohydrate (InhaLac 230) was obtained from Meggle (Wasserburg, Germany). Magnesium stearate was purchased from Macron Chemicals (Radnor, PA). Size 3 hydroxypropyl methylcellulose capsules were a generous gift by Capsugel (Morristown, NJ). 200 proof ethanol was obtained from Decon Labs (King of Prussia, PA), and acetonitrile and methanol from Fisher Chemical (Hampton, NH). All solvents were of analytical grade.

### Micronization

Budesonide was micronized using an Alpine Spiral Jet Mill 50AS (Hosokawa, Summit, NJ). The material was processed using grinding and injector pressures of 3.7 bar and 4.0 bar, respectively. Particle size of milled material was determined using X-ray diffraction (XRD), and particle size was confirmed to be dᵥ50: 1.96 μm.

### Twin-Screw Mixing

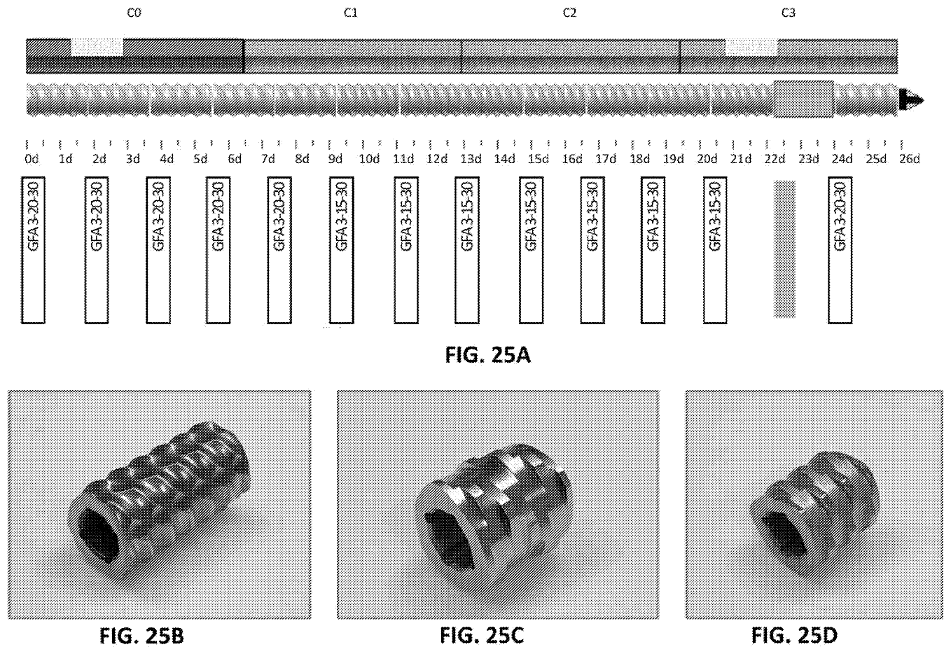

Micronized budesonide, magnesium stearate and lactose were split fed using two volumetric twin-screw feeders (Brabender, Mississauga, Canada). The screw profiles that were used to process the blends are illustrated in FIG. 25A. The first profile included mostly forward conveying elements of varying pitch with one combing element (GFM) near the barrel exit (GFM-Mar. 15, 1930, FIG. 25B). The second profile replaced the GFM element with a 30° forward kneading element (KB-7-3-15-30, FIG. 25C). The third profile replaced the 30° kneading element with a 60° forward kneading element (KB-7-3-15-30-N, FIG. 25D). For each screw profile, combined feed rate (g/min) and screw speed (rpm) were modified to vary specific throughput and specific energy input to the material. The feeding process is the same as in FIG. 12.

A concentrated blend, “Feeder 1,” of 10% budesonide, 3% magnesium stearate and 87% lactose were mixed for 10 minutes using a 2 qt. V-shell (GlobePharma, New Brunswick, NJ) operated at 25 rpm. The Feeder 1 feedstock was loaded into the twin-screw feeder whose inner surfaces were coated with polytetrafluoroethylene (PTFE) to combat sticking. “Feeder 2” contained 100% lactose. Feeder 1 and Feeder 2 were operated at a 1:9 ratio to achieve a final drug load of 1%. A 16 mm twin-screw corotating extruder with a 20:1 L:D ratio (Leistritz, Somerville, NJ) was used to mix the aerosol powders.

The processing parameters are summarized in Table 7.

TABLE 7
Processing conditions used for the screw profiles
containing a combining or a 30° kneading element.

| | Feed | Screw | Number | Zone 1 | Zone 2 | Zone 3 | Drive |
|---|---|---|---|---|---|---|---|
| Screw | rate | speed | of | temperature | temperature | temperature | power |
| profile | (g/min) | (rpm) | passes | (° C.) | (° C.) | (° C.) | (kW) |
| Combing | 15 | 200 | 1 | 25 | 25 | 25 | 2.24 |
| | 25 | 200 | | | | | |
| | 40 | 200 | 3 | | | | |
| | 10 | 300 | 1 | | | | |
| | 40 | 350 | | | | | |
| | 40 | 500 | | | | | |
| | 10 | 500 | | | | | |
| 30° | 15 | 200 | | | | | |
| kneading | 25 | 200 | | | | | |
| | 10 | 300 | | | | | |
| | 40 | 350 | | | | | |
| | 40 | 500 | | | | | |
| | 10 | 500 | | | | | |

The powder from the first 5 minutes of each run was discarded and collection began once the process reached steady state. Equations (1) and (2) were used to calculated specific throughput and specific energy, respectively:

$$Specific throughput = \frac{feed rate}{R P M} \qquad ( 1 )$$

$$Specific energy = \frac{\begin{matrix}KW ( motor rating ) \times \% torque \times \\ \frac{R P {M}_{running}}{R P {M}_{max}} \times 0.97 ( gearbox efficiency )\end{matrix}}{feed rate} \qquad ( 2 )$$

Specific throughput may represent the output per rpm for a process. Specific energy may be the power applied by the motor to the powder being processed. The resulting powder was discharged onto a conveyor belt (Dorner, Hartland, WI) and 30 mg unit dose samples were taken at predetermined locations and time intervals for content uniformity analysis.

### Evaluating Aerosol Performance

Aerosol performance was evaluated using an abbreviated impactor to facilitate rapid analytical screening of the numerous powders resulting from the experimental design. A size 3 hypromellose (HPMC) capsule filled with 30 mg of powder (300 μg drug) was loaded into a high resistance RS01 inhaler (Plastiape S.p.a, Osnago, Italy). 30 mg samples were taken to represent the typical amount contained in one dose. The inhaler was connected in series to a fast-screening impactor and vacuum pump (Copley Scientific, Nottingham, UK). Flow rate was determined according to Equation (3), which relates flow rate (Q) to pressure drop (AP) and device resistance (R):

$$\sqrt{Δ P} = Q R \qquad ( 3 )$$

Device resistance for the RS01 inhaler is estimated as 0.034 kPa^{1/2} (Lmin⁻¹)⁻¹. The 60 L/min insert was selected to target a pressure drop of 4 kPa across the system. Air was pulled for 4 seconds to achieve 4 L inspiratory volume. Drug content was assayed in each section of the impactor and fine particle fraction (FPF) was calculated as the percent of drug recovered from the filter relative to the total mass of drug recovered from the FSI.

### Drug Content Analysis

High-performance liquid chromatography (Waters, Milford, MA) was used to quantify budesonide content. A Luna C18 150×4.6 mm column (Phenomenex, Torrance, CA) was used with a flow rate of 1 mL/min. 30 mg unit dose samples were dissolved in 40:60 ethanol:H₂O and passed through 0.45 μm PTFE filters. Mobile phase included 40:60 H₂O:acetonitrile. A wavelength of 244 nm was used for budesonide detection. Empower software was used to process the chromatographic data (Waters, Milford, MA).

### Particle Size Distribution

Particle size distribution of aerosol powders was obtained using laser diffraction (Sympatec, Clausthal-Zellerfeld, Germany). The HELOS detector was fitted with a RODOS powder disperser and R3 lens (0.9 μm-175 μm). Dispersing conditions were 3 bar pressure and 50% rotation. WINDOX software (Sympatec, Clausthal-Zellerfeld, Germany) was used to determine volume particle size distribution. Since the composition contained 1% drug, results were primarily representative of lactose size distribution.

### Powder Morphology

Aerosol powders were visualized using a scanning electron microscope (SEM) operated in high vacuum mode (FEI, Hillsboro, OR). Powders lightly adhered to carbon tape were sputter coated with a thin layer of gold (Electron Microscopy Sciences, Hatfield, PA).

### Statistical Analysis

JMP (SAS, Cary, NC) was used to conduct statistical analysis. Least-squares regression was used to generate a predictive model and a pooled t-test was used to detect differences between two sets of data. An α-level of 0.05 was considered statistically significant.

### Processing Conditions and Content Uniformity

Table 8 summarizes the parameters used for all processing conditions.

TABLE 8
Torque, specific energy and specific throughput for all processing conditions where all
torque values were kept at less than 5% of machine limits.

| | | | | | Specific | |
|---|---|---|---|---|---|---|
| | | Screw | | | energy | Specific |
| Screw profile | Feed rate (g/min) | speed (rpm) | Number of passes | Torque (Nm) | $\frac{kW}{kg / hr}$ | throughput (g/min/rpm) |
| Combing | 15 | 200 | 1 | 0.54 | 1.24 | 0.075 |
| | 25 | 200 | | 0.71 | 0.98 | 0.125 |
| | 40 | 200 | | 0.64 | 0.55 | 0.2 |
| | 40 | 200 | 2 | 0.91 | 0.79 | 0.2 |
| | 40 | 200 | 3 | 0.95 | 0.82 | 0.2 |
| | 10 | 300 | 1 | 1.03 | 5.33 | 0.033 |
| | 40 | 350 | | 0.83 | 1.26 | 0.114 |
| | 40 | 500 | | 0.96 | 2.06 | 0.08 |
| | 10 | 500 | | 1.13 | 9.72 | 0.02 |
| 30° | 15 | 200 | | 0.76 | 1.74 | 0.075 |
| kneading | 25 | 200 | | 0.88 | 1.22 | 0.125 |
| | 10 | 300 | | 0.85 | 4.41 | 0.033 |
| | 40 | 350 | | 1.03 | 1.55 | 0.114 |
| | 40 | 500 | | 1.08 | 2.33 | 0.08 |
| | 10 | 500 | | 1.18 | 10.15 | 0.02 |

Not intending to be bound by theory, powder may not be collected from the screw profile containing the 60° kneading element as torque may exceed limits which could result in machine shutdown. After the initial blend was discarded and the process reached the steady-state, 30 mg unit dose samples were collected at T=0, 10, 20, and 30 minutes from each stream exiting the left and right screws. It is important to remember that the timepoints indicate samples taken throughout the duration of the manufacturing process, but residence times for individual powders are less than two minutes. This totaled 8 sample locations for each processing condition. Content uniformity was reported as actual amount of drug recovered from each sample relative to the theoretical amount.

Table 9 summarizes mean recovery and % RSD for all processing conditions.

TABLE 9
Uniformity of aerosol powders prepared using the screw profiles containing a combing or
$a 30 ° kneading element . Recover = \frac{actual drug}{theoretical drug} \times 100 ( n = 8 ) .$

| Screw | Feed rate | Screw speed | Number | Mean | |
|---|---|---|---|---|---|
| profile | (g/min) | (rpm) | of passes | recovery (%) | % RSD |
| Combing | 15 | 200 | 1 | 96.2 | 4.9 |
| | 25 | 200 | | 92.4 | 1.7 |
| | 40 | 200 | | 94.5 | 3.5 |
| | 10 | 300 | | 101.4 | 4.7 |
| | 40 | 350 | | 100.8 | 4.4 |
| | 40 | 500 | | 100.2 | 3.9 |
| | 10 | 500 | | 101.6 | 4.8 |
| 30° | 15 | 200 | | 92.4 | 1.6 |
| kneading | 25 | 200 | | 92.2 | 1.1 |
| | 10 | 300 | | 92.3 | 1.8 |
| | 40 | 350 | | 96.8 | 1.6 |
| | 40 | 500 | | 92.3 | 3.0 |
| | 10 | 500 | | 98.5 | 1.6 |

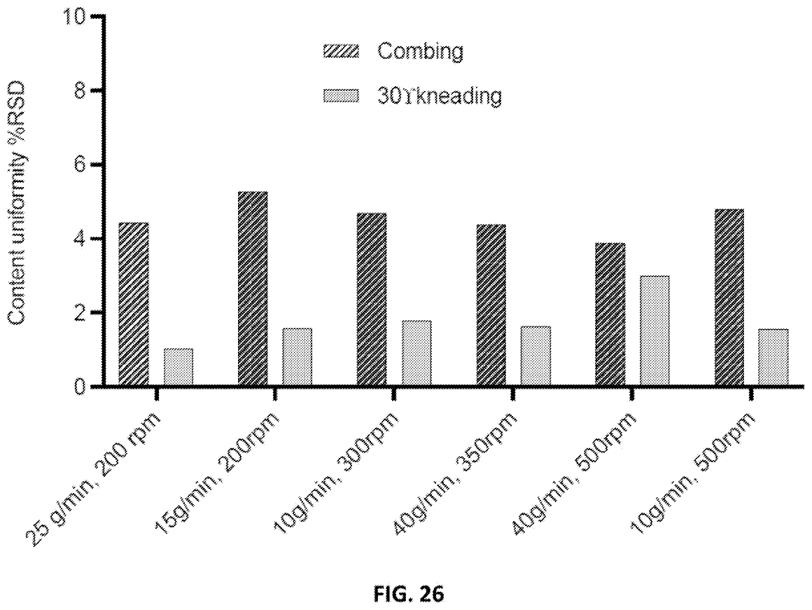

% RSD of drug recovery for all conditions processed using the screw profiles containing the combing or 30° kneading elements are plotted in FIG. 26. All % RSD values are less than 6% and values from the screw profile containing the 30° kneading element are significantly reduced compared to those of the screw profile containing the combing element.

### Example 6: Budesonide Aerosol Performance

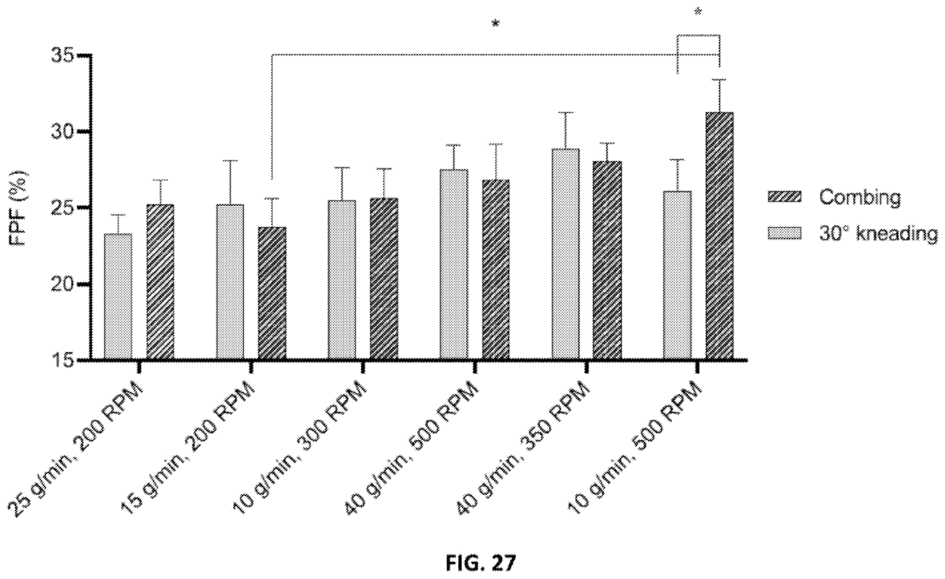

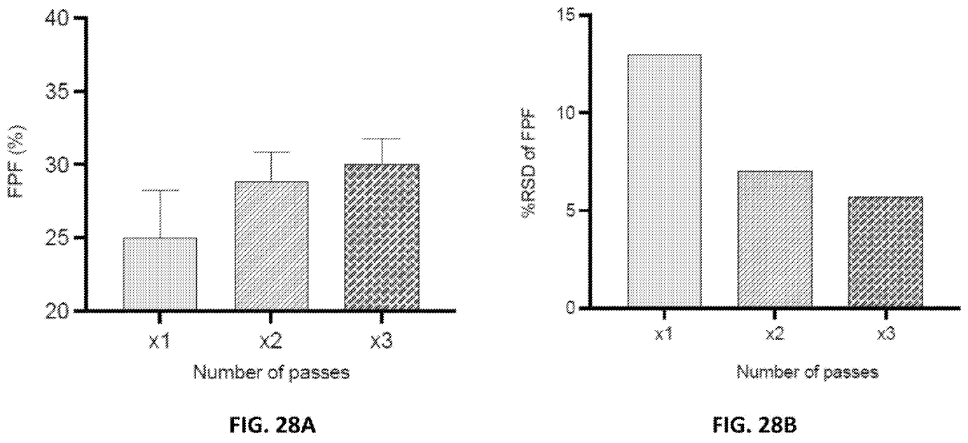

Total drug recovered from the impactor ranged between 90-105%. FIG. 27 compares the aerosol performance of powders produced using both screw profiles. FPF values range from 23.7%-31.3% for the screw profile containing the combing element and 23.3%-27.5% for the screw profile containing the 30° kneading element. There was no significant difference in % FPF between the two screw profiles except for the powders processed at 10 g/min, 500 rpm. The screw profile containing the combing element produced a significantly higher FPF than the screw profile containing the 30° kneading element (31.3% compared to 26.1%). FIG. 28A and FIG. 28B summarize the aerosol performance of powder passed through the twin-screw mixer multiple times at 40 g/min, 200 rpm using the screw profile containing the combing element. Of note, FPF increases while % RSD decreases with each additional pass.

### Particle Size Distribution of Aerosol Powders

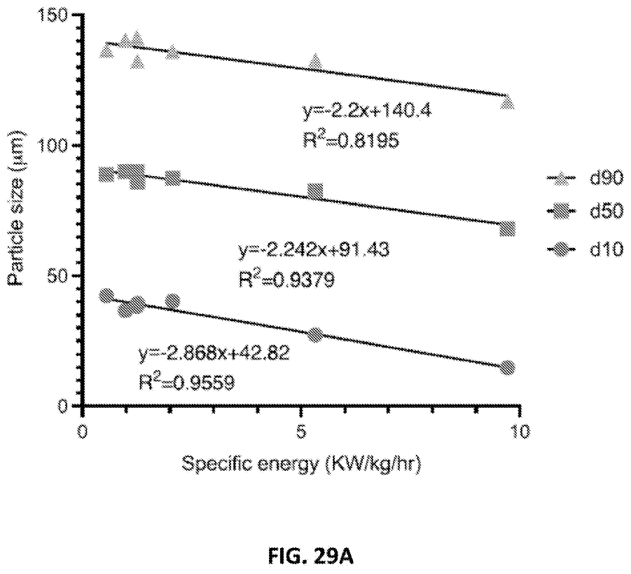

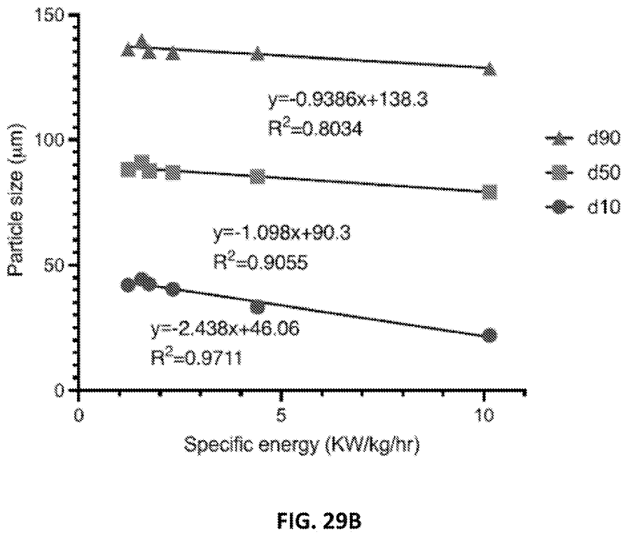

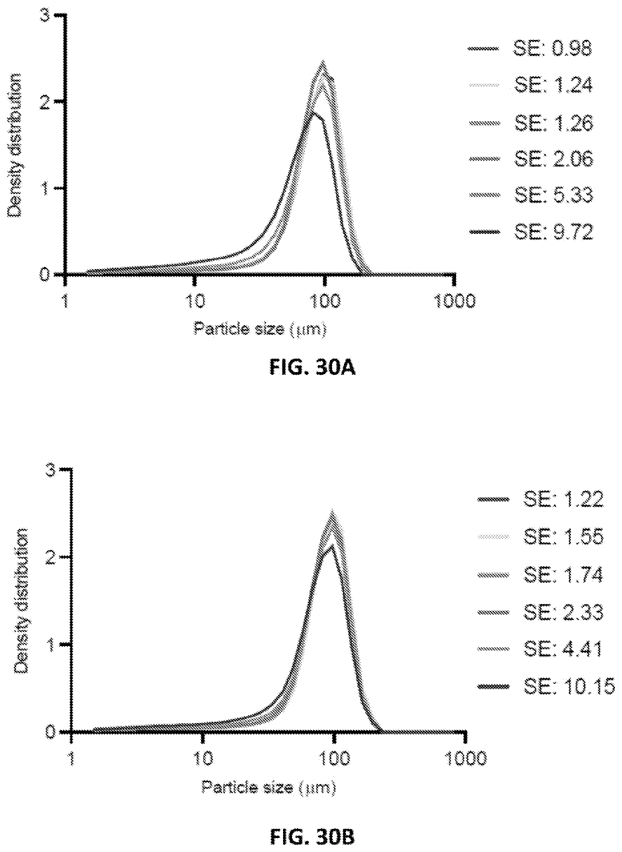

Particle size fractions were plotted as a function of specific energy in FIG. 29A and FIG. 29B. PSD decreases with increasing specific energy for both screw profiles. PSD overlays were plotted for powders produced at each specific energy for both screw profiles (FIG. 30A and FIG. 30B). Span was calculated based on the following equation:

$$Span = \frac{{d}_{v} 90 - {d}_{v} 10}{{d}_{v} 50}$$

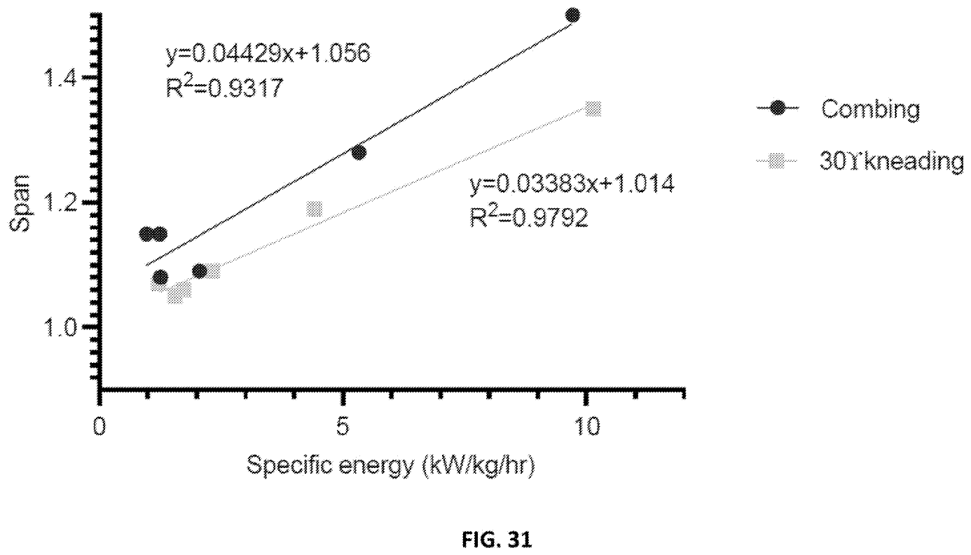

Span was plotted as a function of specific energy for each aerosol powder produced using screw profiles containing either a combing or a 30° kneading element (FIG. 31). Span increases with specific energy, with R2 values of 0.9317 and 0.9792 for the screw profile containing the combing and 30° kneading element, respectively. The span increases at a greater rate for the screw profile containing the combing element.

### Morphology of Aerosol Powders

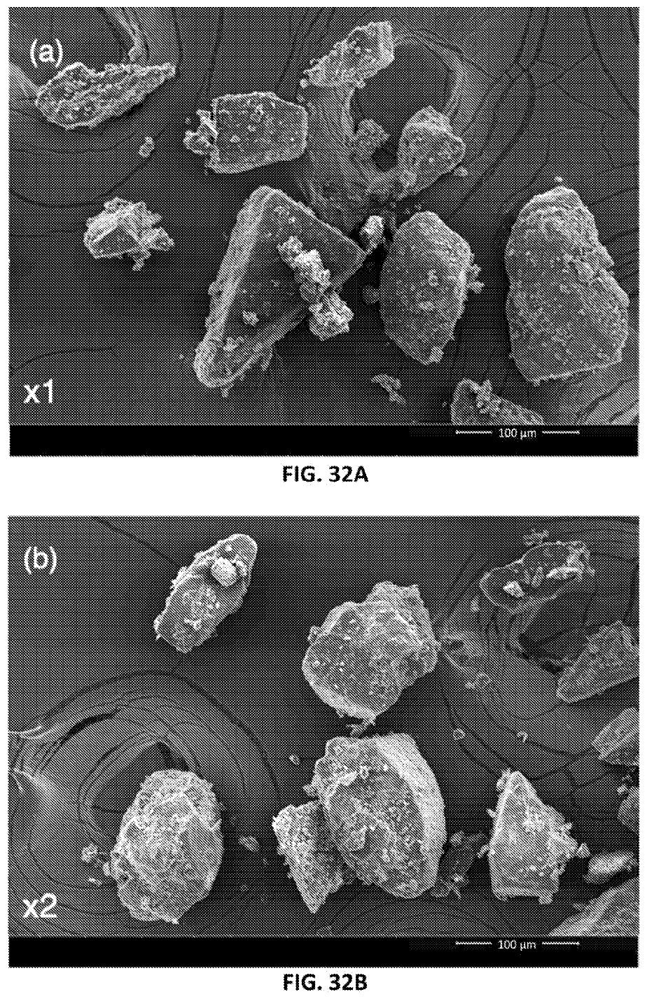

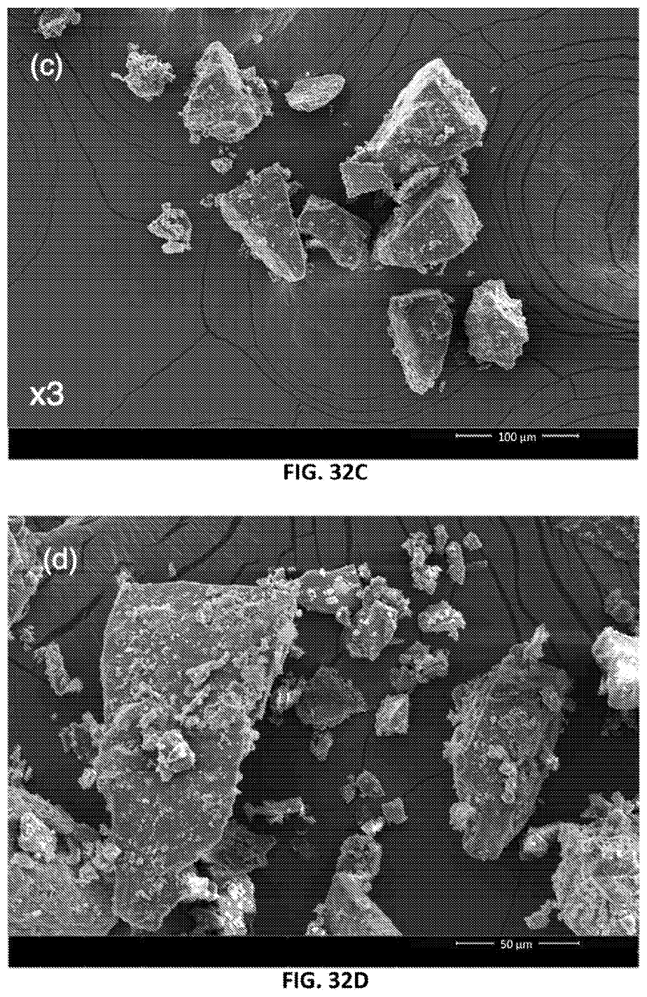

All powders produced using the screw profile containing the combing or 30° kneading element except for 10 g/min, 500 rpm possessed the characteristic tomahawk shape of sieved lactose, with drug particles dispersed on the lactose surface (FIG. 32A-32D). Particle breakage was observed for 10 g/min, 500 rpm powders produced using both screw profiles with a small amount of larger intact particles. The 40 g/min, 200 rpm powder that was passed through the mixer multiple times did not show evidence of particle breakage using SEM.

### Example 7: Twin-Screw Mixing

Jet milling was employed to mechanically reduce the particle size of budesonide to the desired size range suitable for lung delivery. The particle size reduction produces high surface area material with poor flow properties, making it suboptimal for downstream processing and accurate unit dosing. To mitigate these issues, the micronized drug is typically combined with α-lactose monohydrate (dᵥ50:110-150 μm). Not intending to be bound by theory, lactose monohydrate may reduce drug cohesion and improve emptying of the capsule during inhalation. Not intending to be bound by theory, magnesium stearate was also included and may serve as a processing aid and improve aerosolization. Not intending to be bound by theory, a lubricant may doubly serve to weaken lactose and drug interactions to improve aerosol drug delivery.

### Split-Feed Process Design

To assess the capability of the mixer to produce uniform aerosol powders, formulation components were fed into the twin-screw mixer as two separate streams at a 1:9 ratio of 10% drug-lactose mixture in Feeder 1 to lactose in Feeder 2.

Mixing was demonstrated using twin-screw. The mean recovery of the pre-blend was 87.3%±11.1%. A small preblend-to-lactose ratio was used to test the mixing capability of the twin-screw process. All processing conditions evaluated had mean recovery values between 92% and 102%, with less than 5% RSD (Table 9). Not intending to be bound by theory, the plot of content uniformity % RSD in FIG. 26 indicates that the screw profile containing the 30° kneading element produces improved uniformity as compared to the screw profile containing the combing element. For the same feed rate and screw rpm, the mixing intensity increased when processed using the screw profile containing the 30° kneading element compared to the screw profile containing the combing element due to the staggered disc design. These drug fines are then distributed homogeneously throughout the blend by the conveying elements. High shear regions of mixing may be generated in the overflight and intermesh regions, but the screw profile containing the 30° kneading element may have the added benefit of shear regions generated by the wide discs of the kneading element, which may contribute more intensive mixing than the combed mixing element flights.

### Effect of Screw Profile and Processing Conditions

FIG. 27 shows that the % FPF of 10 g/min, 500 rpm blend was significantly higher when processed using the screw profile containing the combing element compared to the screw profile containing the 30° kneading element. For all other processing conditions, the aerosol performance was similar between the two screw profiles. Specific throughput for 10 g/min, 500 rpm is considerably lower (0.02 g/min/rpm) than that of any other condition, for both screw profiles. Not intending to be bound by theory, since specific throughput is inversely proportional to specific energy this indicates 10 g/min, 500 rpm may be the highest specific energy processing condition. The low feed rate combined with the high screw rpm may impart high amounts of shear to the material, which can improve dispersive and distributive mixing. However, with an aggressive screw profile that contains a kneading element, extremely high shear may be detrimental to performance. Cohesive agglomerates require sufficient mixing energy to disperse large particles into smaller units. However, excessive press-on forces may prevent drug and carrier separation during inhalation.

When preparing inhalation powders, high energy input may be beneficial for reducing drug agglomerates to individual particles, which may then be divided and recombined by distributive mixing mechanisms to produce interactive mixtures of drug fines layered onto lactose carrier surface. The mixing intensity may be varied by using screw profiles with different conveying and kneading elements. Not intending to be bound by theory, once below the threshold specific throughput, drug particles may experience adhesion forces greater than the separation forces present during aerosol performance testing and may no longer be separated as fines in the case of the 30° kneading element.

### Effect of Increased Residence Time

The effect of passing the material through the mixer multiple times on aerosol performance was assessed for 40 g/min, 200 rpm using the screw profile containing the combing element. This powder was chosen for analysis due to its high % RSD in FPF after one pass through the mixer (13.0%). After the powder was split-fed into the mixer one time, the material was collected and passed through twice more using one twin-screw feeder. Samples were analyzed using the fast screening impactor (FSI) and budesonide deposition in each part of the impactor was assayed (FIGS. 28A-28B). Comparison of the filter deposition for each pass through the mixer shows an increase in FPF (25% to 30%) and decrease in % RSD of FPF (13% to 6%). Not intending to be bound by theory, results indicate that passing the material through the mixer multiple times simultaneously reduces aerodynamic particle size and variability in filter deposition. With each additional pass, deposition in the induction port decreased, indicating fewer large drug agglomerates were present that are typically filtered out in this section of the impactor (data not shown). Simultaneously, more small particles deposit in the filter. Not intending to be bound by theory, this suggests that agglomerates may be reduced to smaller aerodynamic size fractions with each additional pass through the mixer.

### REFERENCES

Bell, J. H.; Hartley, P. S.; Cox, J. S. G. Dry Powder Aerosols I: A New Powder Inhalation Device. J. Pharm. Sci. 1971, 60 (10), 1559-1564.
Brunaugh, A. D.; Smyth, H. D. C. Formulation Techniques for High Dose Dry Powders. Int. J. Pharm. 2018, 547 (1), 489-498.
Smyth, H. D. C.; Hickey, A. J. Carriers in Drug Powder Delivery. Am. J. Drug Deliv. 2005, 3 (2), 117-132.
Grasmeijer, F.; Hagedoorn, P.; Frijlink, H. W.; de Boer, H. A. Mixing Time Effects on the Dispersion Performance of Adhesive Mixtures for Inhalation. PloS One 2013, 8 (7), e69263.
Bell, J. H.; Hartley, P. S.; Cox, J. S. G. Dry Powder Aerosols I: A New Powder Inhalation Device. J. Pharm. Sci. 1971, 60 (10), 1559-1564.
Smyth, H. D. C.; Hickey, A. J. Carriers in Drug Powder Delivery. Am. J. Drug Deliv. 2005, 3 (2), 117-132.
Jones, M. D.; Santo, J. G. F.; Yakub, B.; Dennison, M.; Master, H.; Buckton, G. The Relationship between Drug Concentration, Mixing Time, Blending Order and Ternary Dry Powder Inhalation Performance. Int. J. Pharm. 2010, 397 (1), 137-147.
Balducci, A. G.; Steckel, H.; Guarneri, F.; Rossi, A.; Colombo, G.; Sonvico, F.; Cordts, E.; Bettini, R.; Colombo, P.; Buttini, F. High Shear Mixing of Lactose and Salmeterol Xinafoate Dry Powder Blends: Biopharmaceutic and Aerodynamic Performances. J. Drug Deliv. Sci. Technol. 2015, 30, 443-449.
Thalberg, K.; Papathanasiou, F.; Fransson, M.; Nicholas, M. Controlling the Performance of Adhesive Mixtures for Inhalation Using Mixing Energy. Int. J. Pharm. 2021, 592, 120055.
Begat, P.; Price, R.; Harris, H.; Morton, D. A. V.; Staniforth, J. N. The Influence of Force Control Agents on the Cohesive-Adhesive Balance in Dry Powder Inhaler Formulations. KONA Powder Part. J. 2005, 23, 109-121.
Burmeister Getz, E.; Carroll, K.; Jones, B.; Benet, L. Batch-to-Batch Pharmacokinetic Variability Confounds Current Bioequivalence Regulations: A Dry Powder Inhaler Randomized Clinical Trial. Clin. Pharmacol. Ther. 2016, 100 (3), 223-231.
Hochhaus, G.; Horhota, S.; Hendeles, L.; Suarez, S.; Rebello, J. Pharmacokinetics of Orally Inhaled Drug Products. AAPS J. 2015, 17 (3), 769-775.
Lee, S. L.; Saluja, B.; García-Arieta, A.; Santos, G. M. L.; Li, Y.; Lu, S.; Hou, S.; Rebello, J.; Vaidya, A.; Gogtay, J.; Purandare, S.; Lyapustina, S. Regulatory Considerations for Approval of Generic Inhalation Drug Products in the US, EU, Brazil, China, and India. AAPS J. 2015, 17 (5), 1285-1304.
Nair, V.; Hefnawy, A.; Smyth, H.; Lee, J.; Feng, K.; Bielski, E.; Dhapare, S.; Newman, B.; Conti, D.; Herpin, M. In Vitro Batch-to-Batch Variability of Fluticasone Propionate and Salmeterol Dry Powder Inhalers; 2021.
Hefnawy, A.; Herpin, M.; Nair, V.; Lee, J.; Feng, K.; Bielski, E.; Dhapare, S.; Newman, B.; Conti, D.; Smyth, H. Investigation of the Effects of Physicochemical Properties of Dry Powder
Inhalers on Batch-to-Batch Variability; 2021. Marek, S. R.; Donovan, M. J.; Smyth, H. D. C. Effects of Mild Processing Pressures on the Performance of Dry Powder Inhaler Formulations for Inhalation Therapy (1): Budesonide and Lactose. Eur. J. Pharm. Biopharm. 2011, 78 (1), 97-106.
Pernenkil, L.; Cooney, C. L. A Review on the Continuous Blending of Powders. Chem. Eng. Sci. 2006, 61 (2), 720-742.
Teżyk, M.; Milanowski, B.; Ernst, A.; Lulek, J. Recent Progress in Continuous and Semi-Continuous Processing of Solid Oral Dosage Forms: A Review. Drug Dev. Ind. Pharm. 2016, 42 (8), 1195-1214.
Nagy, B.; Farkas, A.; Gyürkés, M.; Komaromy-Hiller, S.; Démuth, B.; Szabó, B.; Nusser, D.; Borbás, E.; Marosi, G.; Nagy, Z. K. In-Line Raman Spectroscopic Monitoring and Feedback Control of a Continuous Twin-Screw Pharmaceutical Powder Blending and Tableting Process. Int. J. Pharm. 2017, 530 (1), 21-29.
Fonteyne, M.; Vercruysse, J.; De Leersnyder, F.; Besseling, R.; Gerich, A.; Oostra, W.; Remon, J. P.; Vervaet, C.; De Beer, T. Blend Uniformity Evaluation during Continuous Mixing in a Twin Screw Granulator by In-Line NIR Using a Moving F-Test. Anal. Chim. Acta 2016, 935, 213-223.
Kreimer, M.; Aigner, I.; Lepek, D.; Khinast, J. Continuous Drying of Pharmaceutical Powders Using a Twin-Screw Extruder. Org. Process Res. Dev. 2018, 22 (7), 813-823.
Kittikunakorn, N.; Liu, T.; Zhang, F. Twin-Screw Melt Granulation: Current Progress and Challenges. Int. J. Pharm. 2020, 588, 119670.
Jones, M. D.; Harris, H.; Hooton, J. C.; Shur, J.; King, G. S.; Mathoulin, C. A.; Nichol, K.; Smith, T. L.; Dawson, M. L.; Ferrie, A. R.; Price, R. An Investigation into the Relationship between Carrier-Based Dry Powder Inhalation Performance and Formulation Cohesive-Adhesive Force Balances. Eur. J. Pharm. Biopharm. 2008, 69 (2), 496-507.
Selvam, P.; Smyth, H. D. C. Effect of Press-on Forces on Drug Adhesion in Dry Powder Inhaler Formulations. J. Adhes. Sci. Technol. 2011, 25 (14), 1659-1670.
Saleem, I.; Smyth, H.; Telko, M. Prediction of Dry Powder Inhaler Formulation Performance from Surface Energetics and Blending Dynamics. Drug Dev. Ind. Pharm. 2008, 34 (9), 1002-1010.
Donovan, M. J.; Smyth, H. D. C. Influence of Size and Surface Roughness of Large Lactose Carrier Particles in Dry Powder Inhaler Formulations. Int. J. Pharm. 2010, 402 (1), 1-9.
Yazdi, A. K.; Smyth, H. D. C. Carrier-Free High-Dose Dry Powder Inhaler Formulation of Ibuprofen: Physicochemical Characterization and in Vitro Aerodynamic Performance. Int. J. Pharm. 2016, 511 (1), 403-414.
Yazdi, A. K.; Smyth, H. D. C. Hollow Crystalline Straws of Diclofenac for High-Dose and Carrier-Free Dry Powder Inhaler Formulations. Int. J. Pharm. 2016, 502 (1), 170-180.
Yazdi, A. K.; Smyth, H. D. C. Implementation of Design of Experiments Approach for the Micronization of a Drug with a High Brittle-Ductile Transition Particle Diameter. Drug Dev. Ind. Pharm. 2017, 43 (3), 364-371.
Adams, W. P.; Lee, S. L.; Plourde, R.; Lionberger, R. A.; Bertha, C. M.; Doub, W. H.; Bovet, J.-M.; Hickey, A. J. Effects of Device and Formulation on In Vitro Performance of Dry Powder Inhalers. AAPS J. 2012, 14 (3), 400-409.
Kittikunakorn, N.; Paul, S.; Koleng, J. J.; Liu, T.; Cook, R.; Yang, F.; Bi, V.; Durig, T.; Sun, C. C.; Kumar, A.; Zhang, F. How Does the Dissimilarity of Screw Geometry Impact Twin-Screw Melt Granulation? Eur. J. Pharm. Sci. 2021, 157, 105645.
Jaffari, S.; Forbes, B.; Collins, E.; Barlow, D. J.; Martin, G. P.; Murnane, D. Rapid Characterisation of the Inherent Dispersibility of Respirable Powders Using Dry Dispersion Laser Diffraction. Int. J. Pharm. 2013, 447 (1), 124-131.
Begat, P.; Morton, D. A. V.; Shur, J.; Kippax, P.; Staniforth, J. N.; Price, R. The Role of Force Control Agents in High-Dose Dry Powder Inhaler Formulations. J. Pharm. Sci. 2009, 98 (8), 2770-2783.
Rahimpour, Y.; Hamishehkar, H. Lactose Engineering for Better Performance in Dry Powder Inhalers. Adv. Pharm. Bull. 2012, 2 (2), 183-187.
Agrawal, S.; Ashokraj, Y.; Bharatam, P. V.; Pillai, O.; Panchagnula, R. Solid-State Characterization of Rifampicin Samples and Its Biopharmaceutic Relevance. Eur. J. Pharm. Sci. 2004, 22 (2), 127-144.
Hickey, A.; Concessio, N.; Van Oort, M.; Platz, R. Factors Influencing the Dispersion of Dry Powders as Aerosols. Pharm. Technol. 1994, 18 (8), 58-64.
Sethuraman, V. V.; Hickey, A. J. Powder Properties and Their Influence on Dry Powder Inhaler Delivery of an Antitubercular Drug. AAPS PharmSciTech 2002, 3 (4), 7.
Le, V. N. P.; Thi, T. H. H.; Robins, E.; Flament, M. P. Dry Powder Inhalers: Study of the Parameters Influencing Adhesion and Dispersion of Fluticasone Propionate. AAPS PharmSciTech 2012, 13 (2), 477-484.
Dunber, C. A.; Hickey, A. J.; Holzner, P. Dispersion and Characterization of Pharmaceutical Dry Powder Aerosols. KONA Powder Part. J. 1998, 16, 7-45.
Hertel, M.; Schwarz, E.; Maria Littringer, E.; Dogru, M.; Hauptstein, S.; Steckel, H.; Scherließ, R. Influence of Blender Type on the Performance of Ternary Dry Powder Inhaler Formulations; 2016.
Selvam, P.; Marek, S.; Truman, C. R.; McNair, D.; Smyth, H. D. C. Micronized Drug Adhesion and Detachment from Surfaces: Effect of Loading Conditions. Aerosol Sci. Technol. 2011, 45 (1), 81-87.
Dickhoff, B. H. J.; de Boer, A. H.; Lambregts, D.; Frijlink, H. W. The Effect of Carrier Surface and Bulk Properties on Drug Particle Detachment from Crystalline Lactose Carrier Particles during Inhalation, as Function of Carrier Payload and Mixing Time. Eur. J. Pharm. Biopharm. 2003, 56 (2), 291-302.
Dickhoff, B. H. J.; de Boer, A. H.; Lambregts, D.; Frijlink, H. W. The Interaction between Carrier Rugosity and Carrier Payload, and Its Effect on Drug Particle Redispersion from Adhesive Mixtures during Inhalation. Eur. J. Pharm. Biopharm. 2005, 59 (1), 197-205.
Dickhoff, B. Adhesive Mixtures for Powder Inhalation: The Effect of Carrier (Surface and Bulk) Properties, Carrier Payload and Mixing Conditions on the Performance of Adhesive Mixtures for Inhalation. 2006.
Lavorini, F., Easyhaler ((R)): an overview of an inhaler device for day-to-day use in patients with asthma and chronic obstructive pulmonary disease. Drugs Context, 2019. 8: p. 212596.
van der Palen, J., J. J. Klein, and A. M. Schildkamp, Comparison of a new multidose powder inhaler (Diskus/Accuhaler) and the Turbuhaler regarding preference and ease of use. J Asthma, 1998. 35 (2): p. 147-52.
Fenton, C., G. M. Keating, and G. L. Plosker, Novolizer-A multidose DPI. Drugs, 2003. 63 (22): p. 2437-2445.
Lam, J., S. Vaughan, and M. D. Parkins, Tobramycin Inhalation Powder (TIP): An Efficient Treatment Strategy for the Management of Chronic Pseudomonas Aeruginosa Infection in Cystic Fibrosis. Clin Med Insights Circ Respir Pulm Med, 2013. 7: p. 61-77.
de Boer, A. H. and K. Thalberg, Dry powder inhalers (DPIs), in Inhaled Medicines. 2021. p. 99-146.
de Boer, A. H., H. K. Chan, and R. Price, A critical view on lactose-based drug formulation and device studies for dry powder inhalation: which are relevant and what interactions to expect? Adv Drug Deliv Rev, 2012. 64 (3): p. 257-74.
Heng, P. W. S., L. W. Chan, and L. T. Lim, Quantification of the surface morphologies of lactose carriers and their effect on the in vitro deposition of salbutamol sulphate. Chemical and Pharmaceutical Bulletin, 2000. 48 (3): p. 393-398.
Hertel, M., et al., The influence of high shear mixing on ternary dry powder inhaler formulations. Int J Pharm, 2017. 534 (1-2): p. 242-250.
Sebti, T., F. Vanderbist, and K. Amighi, Evaluation of the content homogeneity and dispersion properties of fluticasone DPI compositions. Journal of Drug Delivery Science and Technology, 2007. 17 (3): p. 223-229.
Burmeister Getz, E., et al., Between-Batch Pharmacokinetic Variability Inflates Type I Error Rate in Conventional Bioequivalence Trials: A Randomized Advair Diskus Clinical Trial. Clin Pharmacol Ther, 2017. 101 (3): p. 331-340.
Spahn, J. E., et al., Development of a novel method for the continuous blending of carrier-based dry powders for inhalation using a co-rotating twin-screw extruder. Int J Pharm, 2022. 623: p. 121914.
Ren, A., et al., Twin-screw continuous mixing can produce dry powder inhalation mixtures for pulmonary delivery. J Pharm Sci, 2022.
Kittikunakorn, N., et al., Effects of thermal binders on chemical stabilities and tabletability of gabapentin granules prepared by twin-screw melt granulation. International Journal of Pharmaceutics, 2019. 559: p. 37-47.
Liu, T., et al., Physicochemical changes and chemical degradation of gliclazide during twin-screw melt granulation. Int J Pharm, 2022. 619: p. 121702.
Keen, J. M., et al., Investigation of process temperature and screw speed on properties of a pharmaceutical solid dispersion using corotating and counter-rotating twin-screw extruders. Journal of Pharmacy and Pharmacology, 2014. 66 (2): p. 204-217.
Rao, R. R., et al., Metamorphosis of Twin Screw Extruder-Based Granulation Technology: Applications Focusing on Its Impact on Conventional Granulation Technology. AAPS PharmSciTech, 2021. 23 (1): p. 24.
Portier, C., et al., Continuous twin screw granulation: A complex interplay between formulation properties, process settings and screw design. Int J Pharm, 2020. 576: p. 119004.
Kumar, A., et al., Mixing and transport during pharmaceutical twin-screw wet granulation: experimental analysis via chemical imaging. Eur J Pharm Biopharm, 2014. 87 (2): p. 279-89.
de Boer, A. H., et al., A critical evaluation of the relevant parameters for drug redispersion from adhesive mixtures during inhalation. Int J Pharm, 2005. 294 (1-2): p. 173-84.
Hrymak, A. N., et al., Polymer Melt Mixing in Twin Screw Extruders, in Reference Module in Materials Science and Materials Engineering. 2016.
Martin, C. et al., Managing Melt Temperature in a Twin Screw Extruder. Leistritz Extrusion, Somerville NJ, 2019.
Brunaugh, A. D., H. D. C. Smyth, and R. O. Williams, Pulmonary Drug Delivery, in Essential Pharmaceutics, 2019, Springer Nature Switzerland AG. p. 163-181.
Huynh, B. K., et al., An Investigation into the Powder Release Behavior from Capsule-Based Dry Powder Inhalers. Aerosol Science and Technology, 2015. 49 (10): p. 902-911.
Telko, M. and A. J. Hickey, Dry Powder Inhaler Formulation. Respiratory Care, 2005. 50 (9).
Timsina, M. P. and G. P. Martin, Drug delivery to the respiratory tract using dry powder inhalers. International Journal of Pharmaceutics, 1993. 101: p. 1-13.
Gramann, P. and C. Rauwendaal New Dispersive and Distributive Mixers for Extrusion and Injection Molding.
Thiele, W., Twin-Screw Extrusion and Screw Design, in Pharmaceutical extrusion technology. 2018. p. 71-93.
Gamble, J. F., et al., Investigation into the degree of variability in the solid-state properties of common pharmaceutical excipients-anhydrous lactose. AAPS PharmSciTech, 2010. 11 (4): p. 1552-7.
Kaialy, W., et al., Influence of lactose carrier particle size on the aerosol performance of budesonide from a dry powder inhaler. Powder Technology, 2012. 227: p. 74-85.
Hertel, N., G. Birk, and R. Scherliess, Can a combination of magnesium stearate and fines tune the inhalable fraction of a mannitol carrier based interactive blend? Drug Delivery to the Lungs, 2019. 30.
Mateo-Ortiz, D., et al., Dry Powder Mixing is Feasible in Continuous Twin Screw Extruder: Towards Lean Extrusion Process for Oral Solid Dosage Manufacturing, AAPS PharmSci Tech (2021) 22:249.

### STATEMENTS REGARDING INCORPORATION BY REFERENCE AND VARIATIONS

All references throughout this application, for example patent documents, including issued or granted patents or equivalents and patent application publications, and non-patent literature documents or other source material are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including.” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A process for making a dry powder inhaler blend, comprising:
introducing an active pharmaceutical ingredient, a lubricant, and a carrier into at least one multi-screw extruder; and
continuously mixing the active pharmaceutical ingredient, the lubricant, and the carrier in the at least one multi-screw extruder to form a dry powder inhaler blend,
wherein the active pharmaceutical ingredient, the lubricant, and the carrier are in the form of a powder.

2. The process of claim 1, wherein the at least one multi-screw extruder comprises at least two co-rotating screws and an intermeshing region.

3. The process of claim 2, wherein the intermeshing region is positioned between the at least two co-rotating screws.

4. The process of claim 1, wherein the multi-screw extruder is operated at a screw speed of rotation of at least 50 rpm.

5. The process of claim 4, wherein the screw speed of rotation is from 50 rpm to 1000 rpm.

6. The process of claim 1, wherein a feed rate of the components into the multi-screw extruder is from 1 g/min to 50 g/min.

7. The process of claim 1, wherein the dry powder inhaler blend has a coefficient of variation percent (% CV) value of less than 5% for any combination of screw speed and feed rate.

8. The process of claim 7, wherein the % CV value is less than 5% where the screw speed is 50 rpm and the feed rate is 10 g/min.

9. The process of claim 7, wherein the % CV value is less than 5% where the screw speed is 100 rpm and the feed rate is 20 g/min.

10. The process of claim 7, wherein the % CV value is less than 5% where the screw speed is 200 rpm and the feed rate is 15 g/min.

11. The process of claim 7, wherein the % CV value is less than 5% where the screw speed is 200 rpm and the feed rate is 25 g/min.

12. The process of claim 7, wherein the % CV value is less than 5% where the screw speed is 200 rpm and the feed rate is 40 g/min.

13. The process of claim 7, wherein the % CV value is less than 5% where the screw speed is 300 rpm and the feed rate is 10 g/min.

14. The process of claim 7, wherein the % CV value is less than 5% where the screw speed is 350 rpm and the feed rate is 40 g/min.

15. The process of claim 7, wherein the % CV value is less than 5% where the screw speed is 500 rpm and the feed rate is 10 g/min.

16. The process of claim 7, wherein the % CV value is less than 5% where the screw speed is 500 rpm and the feed rate is 40 g/min.

17. A capsule for an inhaler, comprising:
a dry powder inhaler blend comprising an active pharmaceutical ingredient, a lubricant, and a carrier,
wherein the dry powder inhaler blend is prepared according to the process of claim 1.

18. The capsule of claim 17, wherein the active pharmaceutical ingredient comprises an agent suitable for treating a pulmonary disease or a pulmonary infection.

19. The capsule of claim 18, wherein the agent suitable for treating a pulmonary disease or a pulmonary infection comprises an antibacterial agent or a steroid.

20. (canceled)

21. (canceled)

22. A dry powder inhaler blend, comprising:
rifampicin or budesonide in an amount of 5 wt. % or less;
a lubricant; and
a carrier,
wherein % CV value is less than 5%.

23.-52. (canceled)
